SEQUENCE LISTING (8 sequences)

SEQ ID NO: 1 - DNA, 81 bp, Artificial Sequence
  Description of Artificial Sequence Synthetic oligonucleotide
tgattctctatacccattatgacctggatattggtattattgtggccatttctacctcatcacacgttctggagaattgtt

SEQ ID NO: 2 - DNA, 19 bp, Artificial Sequence
  Description of Artificial Sequence Synthetic primer
ttgattctctatacccatt

SEQ ID NO: 3 - DNA, 19 bp, Artificial Sequence
  Description of Artificial Sequence Synthetic primer
aacaattctccagaacgtg

SEQ ID NO: 4 - DNA, 138 bp, Artificial Sequence
  Description of Artificial Sequence Synthetic polynucleotide
atttaaacggatagttctgcagcctgaacttaaatgttttcaggataaaacagtttcaaaaatgacttaccgaaaatcttcaacttgtggcaatggaattttggaacctacagagcagtgtgattgtggctataaaga

SEQ ID NO: 5 - DNA, 19 bp, Artificial Sequence
  Description of Artificial Sequence Synthetic primer
atttaaacggatagttctg

SEQ ID NO: 6 - DNA, 19 bp, Artificial Sequence
  Description of Artificial Sequence Synthetic primer
tctttatagccacaatcac

SEQ ID NO: 7 - DNA, 22 bp, Artificial Sequence
  Description of Artificial Sequence Synthetic primer
cctgggctctgtaaagaatagt

SEQ ID NO: 8 - DNA, 21 bp, Artificial Sequence
  Description of Artificial Sequence Synthetic primer
cagagcttaaactgggaagct

(19) United States
(12) Patent Application Publication
(10) Pub. No.: US 2010/0285468 A1
(43) Pub. Date: Nov. 11, 2010

(54) DETECTION AND/OR QUANTIFICATION OF NUCLEIC ACIDS

(75) Inventor: Xing Xin, Foster City, CA (US)

Correspondence Address: WILSON, SONSINI, GOODRICH & ROSATI, 650 PAGE MILL ROAD, PALO ALTO, CA, 94304-1050, US

(73) Assignee: AlleLogic Biosciences Corporation, Hayward, CA (US)

(21) Appl. No.: 12/679,534

(22) PCT Filed: Sep. 24, 2008
(86) PCT No.: PCT/US08/77582

§ 371 Date: Jul. 21, 2010

Related U.S. Application Data
(60) Provisional application No. 60/994,969, filed on Sep. 24, 2007.

(51) Int. Cl. C12Q 1/68

(52) U.S. Cl. 435/6

(57) ABSTRACT

The present invention provides compositions, methods, and kits for nucleic acids analyses. In particular, melting analyses are used to detect the presence or absence and to quantify nucleic acids.

### CROSS-REFERENCE

This application claims the benefit of U.S. Provisional Application No. 60/994,969, filed Sep. 24, 2007, which application is incorporated herein by reference in its entirety.

### BACKGROUND OF THE INVENTION

Nucleic acid analysis is becoming an important tool for the diagnosis and prognosis of infectious as well as genetic diseases. Genetic modifications including variation in gene copy number can lead to profound abnormalities at the cellular and organismal level. Changes in gene copy number may lead to under- or overexpression of genes responsible for a disease phenotype. Other genetic modifications such as chromosomal changes, including allelic loss, mutations, rearrangement, point mutation, deletion, gene amplifications and acquisition of viral genomes have been identified as the hallmark of neoplasia. These changes can result in the loss of tumor suppressor genes and the turning of a cellular proto-oncogene to an actual oncogene. Single copy changes in specific chromosomes or smaller regions can result in a number of developmental disorders, including Down, Prader Willi, Angelman and Cri du chat syndromes. The extra genetic material in these patients causes the phatogenic phenotype associated with these syndromes.

Moreover, variations in the DNA sequences of humans take many forms and can be correlated with how humans develop diseases and respond to pathogens, chemicals, drugs, vaccines, and other agents. For instance, the inheritance of a substantial number of disease traits can be predicted by genetic analysis. Recently, multiple studies have discovered an abundance of submicroscopic copy number variation of DNA segments ranging to kilobases (kb) to megabases (Mb) in size. Deletions, insertions, duplications and complex multi-site variations collectively known as copy number variation regions (CNVR) or copy number polymorphism (CNP). CNVRs can affect gene expression, phenotypic variation and adaptation by disrupting genes and altering gene dosage. In addition, CNVRs can cause disease, as in microdeletion or microduplication disorders, or confer risk to disease traits such as HIV infection and glomerulonephritis. Some CNVRs have been associated with specific diseases such as CHARGE syndrome, Parkinson's and Alzheimer disease.

Current methods for the analysis of cellular genetic content include comparative genomic hybridization (CGH), spectral karyotyping (SKY), fluorescence in situ hybridization (FISH), molecular subtraction methods, such as RDA, digital karyotyping, array based approaches, MLPA and real time PCR. However, these methods are of limited mapping resolution, time consuming and labor intensive. In addition expensive reagents and/or instruments may be required for performing these conventional methods.

### SUMMARY OF THE INVENTION

The invention relates to methods, compositions and devices, e.g., for detecting a target nucleic acid in a sample. The methods of the present invention allows for a rapid cost effective single assay format to determine the presence or absence and/or amount of nucleic acid sequences in a polynucleotide sample.

In some embodiments, the invention provides a method for determining the amount of a target nucleic acid in a sample. In some embodiments, the invention provides a method for determining the amount of a target nucleic acid in a sample containing a nucleic acid binding agent, a target nucleic acid and a reference nucleic acid; where the target nucleic and the reference nucleic acid exhibit distinct melting profiles and where the binding agent yields a detectable signal when bound to the target nucleic acid and/or the reference nucleic acid. The melting profiles of the target nucleic and the reference nucleic acid are determined by detecting the signal of the binding agent at a plurality of temperatures. The amount of target nucleic acid is determined by comparing the melting profile of the target nucleic acid with the melting profile of the reference nucleic acid. In some embodiments, the melting profiles from the target and references nucleic acid are compared by determining the relative amount of signal attributable to the target nucleic acid or to the reference nucleic acid based on their melting profile. In some embodiments, the melting profiles from the target and references nucleic acid are compared by comparing the relative amount of the signal attributable to the target nucleic acid and the reference nucleic acid. In some embodiments, the melting profiles from the target and references nucleic acid are compared by calculating the ratio between the amount of signal attributable to the target nucleic acid and the amount of signal attributable to the reference nucleic acid. The signal can be a fluorescent signal, magnetic signal, radioactive signal, Raman signal or an electrochemical signal.

The target nucleic acid and/or the reference nucleic can be amplified products of the target and/or the reference nucleic acids. In some embodiments, the target nucleic acid and/or the reference nucleic are amplified by a PCR reaction. In some embodiments, the amplified products of the target nucleic acid and the amplified products of the reference nucleic are generated by using a distinct set of primers specific for the target nucleic acid and the reference nucleic acid respectively.

In some embodiments, the target nucleic acid is a genomic DNA region. The genomic DNA region can contain one or more polymorphisms. In some embodiments, the genomic DNA region contains one or more copy number variable regions (CNVR). In some embodiments, the genomic DNA region contains STRs or SNPs. In some embodiments, the target nucleic acid and the reference nucleic acid are double stranded. In some embodiments, the size of the target nucleic acid is about 100 bp to about 1 kilobase. In some embodiments, the target nucleic acid and the reference nucleic acid are comparable in length.

In some embodiments, the reference nucleic acid is a genomic DNA region. In some embodiments, the reference nucleic acid is a cDNA transcript, or an oligonucleotide. In some embodiments, the copy number of the reference nucleic acid is known.

In some embodiments, the target nucleic acid is associated with condition. Examples of conditions include, but are not limited to, trisomy 13, trisomy 18, trisomy 21, Klinefelter Syndrome, dup (17)(p 11.2p 11.2) syndrome, Down syndrome, Pre-eclampsia, Pre-term labor, Edometriosis, Pelizaeus-Merzbacher disease, dup (22)(q11.2q11.2) syndrome, Cat eye syndrome, Cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Kallmann syndrome, microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c), 1p 36 deletion, acute lymphoblastic leukemia, acute or chronic lymphocyctic or granulocytic tumor, acute myeloid leukemia, acute promyelocytic leukemia, adenocarcinoma, adenoma, adrenal cancer, basal cell carcinoma, bone cancer, brain cancer, breast cancer, bronchi cancer, cervical dysplasia, chronic myelogenous leukemia, colon cancer, epidermoid carcinoma, Ewing's sarcoma, gallbladder cancer, gallstone tumor, giant cell tumor, glioblastoma multiforma, hairy-cell tumor, head cancer, hyperplasia, hyperplastic corneal nerve tumor, in situ carcinoma, intestinal ganglioneuroma, islet cell tumor, Kaposi's sarcoma, kidney cancer, larynx cancer, leiomyomater tumor, liver cancer, lung cancer, lymphomas, malignant carcinoid, malignant hypercalcemia, malignant melanomas, marfanoid habitus tumor, medullary carcinoma, metastatic skin carcinoma, mucosal neuromas, mycosis fungoide, myelodysplastic syndrome, myeloma, neck cancer, neural tissue cancer, neuroblastoma, osteogenic sarcoma, osteosarcoma, ovarian tumor, pancreas cancer, parathyroid cancer, pheochromocytoma, polycythemia vera, primary brain tumor, prostate cancer, rectum cancer, renal cell tumor, retinoblastoma, rhabdomyosarcoma, seminoma, skin cancer, small-cell lung tumor, soft tissue sarcoma, squamous cell carcinoma, stomach cancer, thyroid cancer, topical skin lesion, veticulum cell sarcoma, and Wilm's tumor.

In some embodiments, the target nucleic acid is associated with a risk to develop a disease. Examples of diseases include, but are not limited to, HIV infection, glomerulonephritis, CHARGE syndrome, Parkinson's disease and Alzheimer's disease.

In some embodiments, the copy number of the target nucleic acid is determined.

In some embodiments, the nucleic acid binding agent is a DNA binding agent. In some embodiments, the DNA binding agent is a DNA intercalator. Examples of DNA binding agents that can be used in the methods described herein include, but are not limited to, EvaGreen, SYBR Green I, PicoGreen, Cyto 9, LC Green, SYBR GreenER, Ethidium bromide, TOTO, YOYO, Bebo, SYTO Green and bexto. In some embodiments, the DNA binding agent is EvaGreen.

In some embodiments the invention provides a method of determining a genetic condition in a patient or a fetus by analyzing a sample. In some embodiments, the invention provides a method of determining a genetic condition in a patient or a fetus by analyzing a sample suspected to contain a target nucleic acid, a reference nucleic acid and a nucleic acid binding agent. The target nucleic and the reference nucleic acid exhibit distinct melting profiles. The target nucleic acid and the reference nucleic acid are amplified using a first set of primers specific for the target nucleic acid and a second set of primers specific from the reference nucleic acid. The presence or absence of the genetic condition is determined by comparing the melting profile of the amplified target nucleic acid to the melting profile of the amplified reference nucleic acid, where the melting profiles of the amplified target nucleic acid and the amplified reference nucleic acid are determined by monitoring a signal from the binding agent at a plurality of temperatures. In some embodiments, the melting profiles from the target and references nucleic acid are compared by determining the relative amount of signal attributable to the target nucleic acid or to the reference nucleic acid based on their melting profile. In some embodiments, the melting profiles from the target and references nucleic acid are compared by comparing the relative amount of the signal attributable to the target nucleic acid and the reference nucleic acid. In some embodiments, the melting profiles from the target and references nucleic acid are compared by calculating the ratio between the amount of signal attributable to the target nucleic acid and the amount of signal attributable to the reference nucleic acid. The signal can be a fluorescent signal, magnetic signal, radioactive signal, Raman signal or an electrochemical signal.

Examples of conditions include, but are not limited to, trisomy 13, trisomy 18, trisomy 21, Klinefelter Syndrome, dup (17)(p 11.2 p 11.2) syndrome, Down syndrome, Pre-eclampsia, Pre-term labor, Edometriosis, Pelizaeus-Merzbacher disease, dup (22)(q11.2q11.2) syndrome, Cat eye syndrome, Cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Kallmann syndrome, microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c), 1p 36 deletion, acute lymphoblastic leukemia, acute or chronic lymphocyctic or granulocytic tumor, acute myeloid leukemia, acute promyelocytic leukemia, adenocarcinoma, adenoma, adrenal cancer, basal cell carcinoma, bone cancer, brain cancer, breast cancer, bronchi cancer, cervical dysplasia, chronic myelogenous leukemia, colon cancer, epidermoid carcinoma, Ewing's sarcoma, gallbladder cancer, gallstone tumor, giant cell tumor, glioblastoma multiforma, hairy-cell tumor, head cancer, hyperplasia, hyperplastic corneal nerve tumor, in situ carcinoma, intestinal ganglioneuroma, islet cell tumor, Kaposi's sarcoma, kidney cancer, larynx cancer, leiomyomater tumor, liver cancer, lung cancer, lymphomas, malignant carcinoid, malignant hypercalcemia, malignant melanomas, marfanoid habitus tumor, medullary carcinoma, metastatic skin carcinoma, mucosal neuromas, mycosis fungoide, myelodysplastic syndrome, myeloma, neck cancer, neural tissue cancer, neuroblastoma, osteogenic sarcoma, osteosarcoma, ovarian tumor, pancreas cancer, parathyroid cancer, pheochromocytoma, polycythemia vera, primary brain tumor, prostate cancer, rectum cancer, renal cell tumor, retinoblastoma, rhabdomyosarcoma, seminoma, skin cancer, small-cell lung tumor, soft tissue sarcoma, squamous cell carcinoma, stomach cancer, thyroid cancer, topical skin lesion, veticulum cell sarcoma, and Wilm's tumor.

In some embodiments, the condition is a risk to develop a disease. Examples of diseases include, but are not limited to, HIV infection, glomerulonephritis, CHARGE syndrome, Parkinson's disease and Alzheimer's disease.

In some embodiments, the target nucleic acid is a genomic DNA region. The genomic DNA region can contain one or more polymorphisms. In some embodiments, the genomic DNA region contains one or more CNVR.

In some embodiments, the genomic DNA region contains STRs or SNPs. In some embodiments, the target nucleic acid and the reference nucleic acid are double stranded. In some embodiments, the size of the target nucleic acid is about 100 by to about 1 kilobase. In some embodiments, the target nucleic acid and the reference nucleic acid are comparable in length.

In some embodiments, the reference nucleic acid is a genomic DNA region. In some embodiments, the reference nucleic acid is a cDNA transcript, or an oligonucleotide. In some embodiments, the copy number of the reference nucleic acid is known.

In some embodiments, the copy number of the target nucleic acid is determined.

In some embodiments, the nucleic acid binding agent is a DNA binding agent. In some embodiments, the DNA binding agent is a DNA intercalator. Examples of DNA binding agents that can be used in the methods described herein include, but are not limited to, EvaGreen, SYBR Green I, PicoGreen, Cyto 9, LC Green, SYBR GreenER, Ethidium bromide, TOTO, YOYO, Bebo, SYTO Green and bexto. In some embodiments, the DNA binding agent is EvaGreen.

In some embodiments, the invention provides a method of determining copy number variation of a target genomic DNA sequence in a sample. In some embodiments, the invention provides a method of determining copy number variation of a target genomic DNA sequence in a sample potentially containing a target genomic DNA sequence, a reference nucleic acid and a nucleic acid binding agent, where the target genomic DNA sequence and the reference nucleic acid exhibit distinct melting profiles. The target DNA sequence and the reference nucleic acid are amplified using a first set of primers specific for the genomic DNA sequence and a second set of primes specific for the reference nucleic acid sequence. The copy number of the target genomic DNA sequence is determined by comparing the melting profile of the target nucleic acid to the melting profile of the reference nucleic acid, where the melting profiles of the target and reference nucleic acid are determined by monitoring the signal of said binding agent at a plurality of temperatures.

In some embodiments, the melting profiles from the target and references nucleic acid are compared by determining the relative amount of signal attributable to the target nucleic acid or to the reference nucleic acid based on their melting profile. In some embodiments, the melting profiles from the target and references nucleic acid are compared by comparing the relative amount of the signal attributable to the target nucleic acid and the reference nucleic acid. In some embodiments, the melting profiles from the target and references nucleic acid are compared by calculating the ratio between the amount of signal attributable to said target nucleic acid and the amount of signal attributable to said reference nucleic acid. The signal can be a fluorescent signal, magnetic signal, radioactive signal, Raman signal or an electrochemical signal.

In some embodiments, the target nucleic acid and the reference nucleic acid are double stranded. In some embodiments, the size of the target nucleic acid is about 100 by to about 1 kilobase. In some embodiments, the target nucleic acid and the reference nucleic acid are comparable in length.

In some embodiments, the reference nucleic acid is a genomic DNA region. In some embodiments, the reference nucleic acid is a cDNA transcript, or an oligonucleotide. In some embodiments, the copy number of the reference nucleic acid is known.

In some embodiments, the nucleic acid binding agent is a DNA binding agent. In some embodiments, the DNA binding agent is a DNA intercalator. Examples of DNA binding agents that can be used in the methods described herein include, but are not limited to, EvaGreen, SYBR Green I, PicoGreen, Cyto 9, LC Green, SYBR GreenER, Ethidium bromide, TOTO, YOYO, Bebo, SYTO Green and bexto. In some embodiments, the DNA binding agent is EvaGreen.

In some embodiments, the invention provides a kit. In some embodiments, the kit contains a nucleic acid binding agent, a set of primers specific for a reference nucleic acid or alternatively a reference nucleic acid; and instructions for the use of the nucleic acid binding agent, and the set of primers or the reference nucleic acid to perform any of the methods described herein. In some embodiments, the kit further contains a set of primers specific for a target nucleic acid. In some embodiments, the kit contains a polymerase. In some embodiments, the kit contains instructions on how to perform amplification of a target nucleic acid and optionally the reference nucleic acid using the polymerase. In some embodiments, the kit contains one or more buffers.

In some embodiments, the reference nucleic acid is a genomic DNA region, a cDNA transcript, or an oligonucleotide. In some embodiments, the nucleic acid binding agent is a DNA binding agent. In some embodiments, the DNA binding agent is a DNA intercalator. Examples of DNA binding agents that can be used in the methods described herein include, but are not limited to, EvaGreen, SYBR Green I, PicoGreen, Cyto 9, LC Green, SYBR GreenER, Ethidium bromide, TOTO, YOYO, Bebo, SYTO Green and bexto. In some embodiments, the DNA binding agent is EvaGreen.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of a melting curve for a reference (peak 1) and a target DNA (peak 2) in which the x-axis is the temperature (T) and the y-axis is −dF/dT, where F is the fluorescence intensity recorded. Various methods of determining the peak ratio for calculation of the amount of the test DNA are also shown.

FIG. 2 shows control results from the copy number determination using the method of ΔΔCt from a real-time PCR assay. The control results are in agreement with previously reported copy numbers.

FIG. 3 shows melting curves obtained from melting analyses of various DNA mix ratios ranging from 1:1 to 1:10 of reference DNA to target DNA.

FIG. 4 shows a standard response curve plotting the peak-height ratio against the input DNA mix ratio in a double log plot. The peak-height ratio is linearly correlated with the input DNA mix ratio.

FIG. 5 shows a standard response curve and demonstrates that a different annealing time during amplification affects the value of the peak-height ratio but does not affect the linear correlation between the peak-height ratio and the input DNA mix ratio.

FIG. 6 shows that the total starting DNA quantity during amplification affects the peak-height ratio but does not affect the linear correlation between the peak-height ratio and the input DNA mix ratio.

FIG. 7 shows a table illustrating how the copy number of a target chromosome region was determined.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entirety.

As used in this application, the singular form “a,” “an,” and “the” include plural references unless the context clearly dictates otherwise. For example, the term “an agent” includes a plurality of agents, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. All references to the function log default to 10 as the base.

The assay of the present invention is particularly useful for analyzing nucleic acids (e.g., DNA, RNA, or hybrids thereby). The methods and compositions described herein provide a sensitive, cost and labor effective assay for determining the presence or absence and/or amount of a target nucleic acid, e.g., the presence or absence and or copy number of a CNVR. The methods described herein typically involve a melting analysis to detect and quantify a target nucleic acid. In some embodiments, a target nucleic acid and a reference nucleic acid that exhibit different melting profiles are amplified using a different set of primers. That is, a first set of primer specific for the target nucleic acid and a second set of primers specific for a reference nucleic acid are used for the amplification. The reference nucleic acid can come from the same or a different source as the target nucleic acid. The melting profiles of the target nucleic acid and reference nucleic acid are determined and compared to deduce the amount of said target nucleic acid. Thus, in some embodiments, melting analysis is used to quantify at least two amplicons that are co-amplified in an amplification reaction. In some cases, the methods described herein are used for the determination of nucleic acid copy number. Methods of using Algorithms and computer software programs that perform the methods described herein are also disclosed.

An individual is not limited to a human being but may also be other organisms including but not limited to mammals, plants, bacteria, or cells derived from any of the above.

Nucleic acids, including target nucleic acids and reference nucleic acids, of the present invention may include any polymer or oligomer of pyrimidine and purine bases, preferably cytosine, thymine, and uracil, and adenine and guanine, either deoxyribonucleotides or ribonucleotides, or analogs thereof. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. Nucleic acids may have any three-dimensional structure, and may perform any function, known or unknown. The following are non-limiting examples of nucleic acids: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A nucleic acid may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A nucleic acid may be further modified after polymerization, such as by conjugation with a labeling component.

An oligonucleotide or polynucleotide is a nucleic acid ranging from about at least 3, 5, 10, or 20 nucleotides in length, but may be up to 100, 1000, or 10,000 nucleotides long or even longer. Polynucleotides of the present invention include sequences of deoxyribonucleic acid (DNA) or ribonucleic acid (RNA) or mimetics thereof which may be isolated from natural sources, recombinantly produced or artificially synthesized. The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

“Genome” designates or denotes the complete, single-copy set of genetic material for an organism. It can be either DNA or RNA. A genome may be multi-chromosomal such that the DNA is cellularly distributed among a plurality of individual chromosomes. For example, in human there are 22 pairs of chromosomes plus a gender associated XX or XY pair.

The term “chromosome” refers to the heredity-bearing gene carrier of a living cell which is derived from chromatin and which comprises DNA and protein components (e.g., histones). The conventional internationally recognized individual human genome chromosome numbering system is employed herein. The size of an individual chromosome can vary from one type to another with a given multi-chromosomal genome and from one genome to another. In the case of the human genome, the entire DNA mass of a given chromosome is usually greater than about 100,000,000 bp. For example, the size of the entire human genome is about 3×10⁹ bp. The largest chromosome, chromosome no. 1, contains about 2.4×10⁸ by while the smallest chromosome, chromosome no. 22, contains about 5.3×10⁷ bp.

A “chromosomal region” is a portion of a chromosome. A “genomic region” is a portion of a genome. The actual physical size or extent of any individual chromosomal or genomic region can vary greatly. The term “region” is not necessarily definitive of any particular one or more genes because a region need not take into specific account the particular coding segments (exons) of an individual gene.

In practicing aspects of the present invention (e.g., performing amplification reactions, etc.), many conventional techniques in molecular biology and recombinant DNA are optionally utilized. These techniques are well known and are explained in, for example, Current Protocols in Molecular Biology, Volumes I, II, and III, 1997 (F. M. Ausubel ed.); Sambrook et al., 2001, Molecular Cloning: A Laboratory Manual, Third Edition, Cold Spring Harbor Laboratory Press, Cold Spring Harbor, N.Y.; Berger and Kimmel, Guide to Molecular Cloning Techniques Methods in Enzymology volume 152 Academic Press, Inc., San Diego, Calif. (Berger), DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); Methods in Enzymology Vol. 154 and Vol. 155 (Wu and Grossman, and Wu, eds., respectively); PCR 2: A PRACTICAL APPROACH (M. J. MacPherson, B. D. Hames and G. R. Taylor eds. (1995)).

In some embodiments target nucleic acids are from a sample obtained from an animal. Such animal can be a human, a domesticated or a laboratory model animal such as a mouse, cow, chicken, drosophila, pig, horse, rabbit, zebra fish, dog, cat, or goat. In some embodiments target nucleic acids are from a sample obtained from a bacteria or virus. Samples derived from an animal, e.g., human, can include, for example whole blood, sweat, tears, ear flow, sputum, lymph, bone marrow suspension, lymph, urine, saliva, semen, vaginal flow, cerebrospinal fluid, brain fluid, ascites, milk, secretions of the respiratory, intestinal or genitourinary tracts fluid. In some embodiments the sample is a cell sample, which can be a primary cell sample or cultivated cell sample or a progeny thereof. Cell samples can be obtained from a variety of tissues depending on the age and condition of the animal. Cell samples can be obtained from peripheral blood using well known techniques. In fetal testing, a sample can be obtained by amniocentesis, chorionic villi sampling or by isolating fetal cells from the blood of a pregnant individual. Other sources of nucleic acids include blood, semen, buccal cells, or the like. Nucleic acids can be obtained from any tissue or organ by methods well known in the art.

In any of the embodiments herein, target nucleic acids are obtained from a single cell.

To obtain a blood sample, any technique known in the art may be used, e.g. a syringe or other vacuum suction device. A blood sample can be optionally pre-treated or processed prior to enrichment. Examples of pre-treatment steps include the addition of a reagent such as a stabilizer, a preservative, a fixant, a lysing reagent, a diluent, an anti-apoptotic reagent, an anti-coagulation reagent, an anti-thrombotic reagent, magnetic property regulating reagent, a buffering reagent, an osmolality regulating reagent, a pH regulating reagent, and/or a cross-linking reagent.

When a blood sample is obtained, a preservative such an anti-coagulation agent and/or a stabilizer can be added to the sample prior to enrichment. This allows for extended time for analysis/detection. Thus, a sample, such as a blood sample, can be analyzed under any of the methods and systems herein within 1 week, 6 days, 5 days, 4 days, 3 days, 2 days, 1 day, 12 hrs, 6 hrs, 3 hrs, 2 hrs, or 1 hr from the time the sample is obtained.

In some embodiments, a blood sample can be combined with an agent that selectively lyses one or more cells or components in a blood sample. For example, fetal cells can be selectively lysed releasing their nuclei when a blood sample including fetal cells is combined with deionized water. Such selective lysis allows for the subsequent enrichment of fetal nuclei using, e.g., size or affinity based separation. In another example platelets and/or enucleated red blood cells are selectively lysed to generate a sample enriched in nucleated cells, such as fetal nucleated red blood cells (fnRBC) and maternal nucleated blood cells (mnBC). The fnRBC's can subsequently be separated from the mnBC's using, e.g., affinity to antigen-i or magnetism differences in fetal and adult hemoglobin.

When obtaining a sample from an animal (e.g., blood sample), the amount can vary depending upon animal size, its gestation period, and the condition being screened. In some embodiments, up to 50, 40, 30, 20, 10, 9, 8, 7, 6, 5, 4, 3, 2, or 1 mL of a sample is obtained. In some embodiments, 1-50, 2-40, 3-30, or 4-20 mL of sample is obtained. In some embodiments, more than 5, 10, 15, 20, 25, 30, 35, 40, 45, 50, 55, 60, 65, 70, 75, 80, 85, 90, 95 or 100 mL of a sample is obtained.

Nucleic acids from samples that can be analyzed by the methods herein include: double-stranded DNA, single-stranded DNA, single-stranded DNA hairpins, DNA/RNA hybrids, RNA (e.g. mRNA or miRNA) and RNA hairpins.

Where desired, nucleic acid contained in the aforementioned samples can be first extracted according to standard methods in the art. For instance, in DNA or RNA can be isolated using various lytic enzymes or chemical solutions according to the procedures set forth in Sambrook et al. (1989), supra or extracted by nucleic-acid-binding resins following the accompanying instructions provided by manufactures.

In some cases, sample analyses involves performing one or more genetic analyses or detection steps on nucleic acids from the enriched product (e.g., enriched cells or nuclei). Nucleic acids from enriched cells or enriched nuclei that can be analyzed by the methods herein include: double-stranded DNA, single-stranded DNA, single-stranded DNA hairpins, DNA/RNA hybrids, RNA (e.g. mRNA) and RNA hairpins Examples of genetic analyses that can be performed on enriched cells or nucleic acids include, e.g., SNP detection, STR detection, and RNA expression analysis.

In some embodiments, less than 1 pg, 5 pg, 10 pg, 20 pg, 30 pg, 40 pg, 50 pg, 100 pg, 200 pg, 500 pg, 1 ng, 5 ng, 10 ng, 20 ng, 30 ng, 40 ng, 50 ng, 100 ng, 200 ng, 500 ng, 1 ug, 5 ug, 10 ug, 20 ug, 30 ug, 40 ug, 50 ug, 100 ug, 200 ug, 500 ug or 1 mg of nucleic acids are obtained from the sample for further genetic analysis. In some cases, about 1-5 pg, 5-10 pg, 10-100 pg, 100 pg-1 ng, 1-5 ng, 5-10 ng, 10-100 ng, 100 ng-1 ug of nucleic acids are obtained from the sample for further genetic analysis.

In some embodiments, the methods described herein are used to detect and/or quantified a target nucleic acid molecule. In some embodiments, the methods described herein are used to detect and/or quantified multiple target nucleic acid molecules. The methods described herein can analyzed at least 1, 2, 3, 4, 5, 10, 20, 50, 100, 200, 500, 1,000, 2,000, 5,000, 10,000, 20,000, 50,000, 100,000, different target nucleic acids.

In some embodiments, the methods described herein are used to detect and/or quantified a target nucleic acid molecule which is about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 400, 410, 420, 430, 440, 450, 460, 470, 480, 490, 500, 510, 520, 530, 540, 550, 560, 570, 580, 590, 600, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 by in length. In some embodiments, the methods described herein are used to detect and/or quantified a target nucleic acid molecule which larger that 1 kb in length. In some embodiments, the methods described herein are used to detect and/or quantified a target nucleic acid molecule which is about 1, 2, 3, 4, 5, 6, 7, 8, 9, 10 Kb in length.

In some embodiments, the methods described herein are used to detect allelic loss, point mutations, deletions, amplifications, or rearrangement in cells from an individual. Normal cells that are heterozygous at one or more loci may give rise to tumor cells that are homozygous at those loci. This loss of heterozygosity may result from structural deletion of normal genes or loss of the chromosome carrying the normal gene, mitotic recombination between normal and mutant genes, followed by formation of daughter cells homozygous for deleted or inactivated (mutant) genes; or loss of the chromosome with the normal gene and duplication of the chromosome with the deleted or inactivated (mutant) gene.

A homozygous deletion is a deletion of both copies of a gene or of a genomic region. Diploid organisms generally have two copies of each autosomal chromosome and therefore have two copies of any selected genomic region. If both copies of a genomic region are absent the cell or sample has a homozygous deletion of that region. Similarly, a hemizygous deletion is a deletion of one copy of a gene or of a genomic region.

Genetic rearrangement occurs when errors occur in DNA replication and cross over occurs between nonhomologous regions resulting in genetic material moving from one chromosomal location to another. Rearrangement may result in altered expression of the genes near the rearrangement.

In some embodiments, the methods described herein are used to detect and/or quantify target nucleic acids to profile a specific tissue or a specific condition. In some embodiments, the methods described herein are used to detect and/or quantify target nucleic acids to detect biomarkers for specific tissue or condition. In some embodiments the methods described herein are used to determine the copy number of a target nucleic acid for specific tissue or condition. In some embodiments, the methods described herein are used to detect and/or quantify target nucleic acids to profile a neoplastic and/or cancer cell. In some embodiments, the methods described herein are used to detect and/or quantify target nucleic acids to diagnose cancer and/or a neoplastic condition. In some embodiments, the methods described herein are used to detect and/or quantify target nucleic acids to detect biomarkers in a neoplastic and/or cancer cell. In some embodiments the methods described herein are used to determine the copy number of a target nucleic acid in a neoplastic and/or cancer cell.

As used herein the term “diagnose” or “diagnosis” of a condition includes predicting or diagnosing the condition, determining predisposition to the condition, monitoring treatment of the condition, diagnosing a therapeutic response of the disease, and prognosis of the condition, condition progression, and response to particular treatment of the condition.

Conditions in a patient that can be detected using the systems and methods herein include, but are not limited to, infection (e.g., bacterial, viral, or fungal infection), neoplastic or cancer conditions (e.g., acute lymphoblastic leukemia, acute or chronic lymphocyctic or granulocytic tumor, acute myeloid leukemia, acute promyelocytic leukemia, adenocarcinoma, adenoma, adrenal cancer, basal cell carcinoma, bone cancer, brain cancer, breast cancer, bronchi cancer, cervical dysplasia, chronic myelogenous leukemia, colon cancer, epidermoid carcinoma, Ewing's sarcoma, gallbladder cancer, gallstone tumor, giant cell tumor, glioblastoma multiforma, hairy-cell tumor, head cancer, hyperplasia, hyperplastic corneal nerve tumor, in situ carcinoma, intestinal ganglioneuroma, islet cell tumor, Kaposi's sarcoma, kidney cancer, larynx cancer, leiomyomater tumor, liver cancer, lung cancer, lymphomas, malignant carcinoid, malignant hypercalcemia, malignant melanomas, marfanoid habitus tumor, medullary carcinoma, metastatic skin carcinoma, mucosal neuromas, mycosis fungoide, myelodysplastic syndrome, myeloma, neck cancer, neural tissue cancer, neuroblastoma, osteogenic sarcoma, osteosarcoma, ovarian tumor, pancreas cancer, parathyroid cancer, pheochromocytoma, polycythemia vera, primary brain tumor, prostate cancer, rectum cancer, renal cell tumor, retinoblastoma, rhabdomyosarcoma, seminoma, skin cancer, small-cell lung tumor, soft tissue sarcoma, squamous cell carcinoma, stomach cancer, thyroid cancer, topical skin lesion, veticulum cell sarcoma, or Wilm's tumor), inflammation, etc.

In some embodiments, the methods described herein are used to distinguish between target nucleic acids that differ from another nucleic acid by a single nucleotide. In some embodiments, the methods described herein are used to distinguish between target nucleic acids that differ from another nucleic acid by 1 nucleotide (nt) or more than 1, 2, 3, 5, 10, 15, 20, 21, 22, 24, 25, 30 or more nucleotides.

In some embodiments, the methods described herein are used to detect and/or quantify target nucleic acids without the need of target nucleic acid isolation. In some embodiments, the methods described herein are used to detect and/or quantify a target nucleic acid directly from a nucleic acid sample comprising DNA and RNA molecules.

The methods described herein can be used to detect and/or quantify genomic DNA regions. The method described herein can be used to detect the copy number of one or more genomic DNA regions. In some embodiments, the methods described herein are used to diagnose a fetal abnormality. Aneuploidy means the condition of having less than or more than the normal diploid number of chromosomes. In other words, it is any deviation from euploidy. Aneuploidy includes conditions such as monosomy (the presence of only one chromosome of a pair in a cell's nucleus), trisomy (having three chromosomes of a particular type in a cell's nucleus), tetrasomy (having four chromosomes of a particular type in a cell's nucleus), pentasomy (having five chromosomes of a particular type in a cell's nucleus), triploidy (having three of every chromosome in a cell's nucleus), and tetraploidy (having four of every chromosome in a cell's nucleus). Birth of a live triploid is extraordinarily rare and such individuals are quite abnormal, however triploidy occurs in about 2-3% of all human pregnancies and appears to be a factor in about 15% of all miscarriages. Tetraploidy occurs in approximately 8% of all miscarriages. (http://www.emedicine.com/med/topic3241.htm).

In some embodiments, the methods described herein are used to detect and/or quantify genomic DNA regions to diagnose a fetal condition such as aneuploidy. In some embodiments, the methods described herein are used to diagnose a fetal abnormality by quantifying a DNA region chosen on a chromosome suspected of aneuploidy and on a control chromosome. In some embodiment aneuploidy is trisomy selected from the group consisting of: trisomy 13, trisomy 18, trisomy21 (Down Syndrome), Klinefelter Syndrome (X X Y), or other irregular number of sex or autosomal chromosomes, and a combination thereof. Examples of chromosomes that are often trisomic include chromosomes 21, 18, 13, and X. In some cases, 1 or more than 1, 2, 3, 4, 5, 6, 7, 8, 9, 10, 15, or 20 regions are detected and quantified per chromosome tested. In some embodiments, the methods described herein can discriminate and quantitate genomic DNA regions. The methods described herein can discriminate and quantitate genomic DNA regions of at least 1, 2, 3, 4, 5, 10, 20, 50, 100, 200, 500, 1,000, 2,000, 5,000, 10,000, 20,000, 50,000, 100,000, different genomic DNA regions. The methods described herein can discriminate and quantitate genomic DNA regions varying by 1 nt or more than 1, 2, 3, 5, 10, 15, 20, 21, 22, 24, 25, 30 nt.

Fetal conditions that can be determined based on the methods and systems herein include the presence of a fetus and/or a condition of the fetus such as fetal aneplouidy e.g., trisomy 13, trisomy 18, trisomy 21 (Down Syndrome), Klinefelter Syndrome (XXY) and other irregular number of sex or autosomal chromosomes. Other fetal conditions that can be detected using the methods herein include segmental aneuploidy, such as 1p 36 duplication, dup (17)(p 11.2 p 11.2) syndrome, Down syndrome, Pelizaeus-Merzbacher disease, dup (22)(q11.2q11.2) syndrome, Cat eye syndrome. In some embodiment, the fetal abnormality to be detected is due to one or more deletions in sex or autosomal chromosomes, including Cri-du-chat syndrome, Wolf-Hirschhorn syndrome, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, steroid sulfatase deficiency, Kallmann syndrome, Microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c) and 1p 36 deletion. In some cases, the fetal abnormality is an abnormal decrease in chromosomal number, such as XO syndrome.

The methods described herein can be used to detect and/or quantify genomic DNA regions such as a region containing a DNA polymorphism. A polymorphism refers to the occurrence of two or more genetically determined alternative sequences or alleles in a population. A polymorphic marker or site is the locus at which divergence occurs. Preferred markers have at least two alleles, each occurring at a frequency of preferably greater than 1%, and more preferably greater than 10% or 20% of a selected population. A polymorphism may comprise one or more base changes, an insertion, a repeat, or a deletion. A polymorphic locus may be as small as one base pair. Polymorphic markers include single nucleotide polymorphisms (SNP's), restriction fragment length polymorphisms (RFLP's), variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens. In some embodiments, the methods described herein can discriminate and quantitate a DNA region containing a DNA polymorphisms. The methods described herein can discriminate and quantitate DNA polymorphism of at least 1, 2, 3, 4, 5, 10, 20, 50, 100, 200, 500, 1,000, 2,000, 5,000, 10,000, 20,000, 50,000, 100,000, different genomic DNA regions.

The methods described herein can be used to detect and/or quantify genomic DNA regions such as copy number variable region (CNVR). Until recently, the importance of large-scale copy number changes in the genomes of humans and other vertebrates has been under-appreciated. Two reports using comparative genomic hybridization with DNA microarrays (array-CGH), highlighted the widespread nature of this normal copy number variation (Iafrate et al. 2004; Sebat et al. 2004). Other studies have now confirmed and further detailed the extent of copy number variation (CNV) in human and primate genomes (Newman et al. 2005; Sharp et al. 2005; Tuzun et al. 2005; Conrad et al. 2006; Perry et al. 2006). CNVR are copy number variation of DNA segments ranging to kilobases (kb) to megabases (Mb) in size, including deletions, insertions, duplications and complex multi-site variants. CNVRs can affect gene expression, phenotypic variation and adaptation by disrupting genes and altering gene dosage. In addition, CNVRs can cause disease, as in microdeletion or microduplication disorders, or confer risk to disease traits such as HIV infection and glomerulonephritis. Some CNVRs have been associated with specific diseases such as CHARGE syndrome, Parkinson's and Alzheimer disease.

In some embodiments, the methods described herein are used to detect and/or quantified a CNVR molecule. In some embodiments, the methods described herein are used to detect and/or quantified multiple CNVR molecules. In some embodiments, the methods described herein are use to determine the copy number of one or more CNVRs. The methods described herein can analyzed at least 1, 2, 3, 4, 5, 10, 20, 50, 100, 200, 500, 1,000, 2,000, 5,000, 10,000, 20,000, 50,000, 100,000, different CNVRs.

The methods described herein can be used to detect and/or quantify CNVRs to profile a specific tissue or a specific condition. In some embodiments, the methods described herein are used to detect and/or quantify CNVRs to detect biomarkers for specific tissue or condition. In some embodiments, the methods described herein are used to detect and/or quantify CNVRs to profile a neoplastic and/or cancer cell. In some embodiments, the methods described herein are used to detect and/or quantify CNVRs to diagnose cancer and/or a neoplastic condition. In some embodiments, the methods described herein are used to detect and/or quantify CNVRs to detect biomarkers in a neoplastic and/or cancer cell.

In some embodiments, the methods described herein are used to detect and/or quantify genomic DNA regions to diagnose a fetal condition such as any disorder or condition associated with CNVRs. Such disorders include, but are not limited to, Parkinson's disease, Alzheimer's disease, dementia, an autism spectrum disorder, susceptibility to viral infection such as HIV, and CHARGE Syndrome. Autism spectrum disorders include Asperger syndrome, autism, PDD not otherwise specified, and Rett disorder. Other known disorders related to CNVRs include, but are not limited to, 12q14 microdeletion syndrome, 15q13.3 microdeletion syndrome, 15q24 recurrent microdeletion syndrome, 16 p 11.2-p 12.2 microdeletion syndrome, 17q21.3 microdeletion syndrome, 1p 36 microdeletion syndrome, 1q21.1 recurrent microdeletion, 1q21.1 recurrent microduplication, 1q21.1 susceptibility locus for Thrombocytopenia-Absent Radius (TAR) syndrome, 22q11 deletion syndrome (Velocardiofacial/DiGeorge syndrome), 22q11 duplication syndrome, 22q11.2 distal deletion syndrome, 22q13 deletion syndrome (Phelan-Mcdermid syndrome), 2 p 15-16.1 microdeletion syndrome, 2q33.1 deletion syndrome, 2q37 monosomy, 3q29 microdeletion syndrome, 3q29 microduplication syndrome, 6p deletion syndrome, 7q11.23 duplication syndrome, 8p 23.1 deletion syndrome, 9q subtelomeric deletion syndrome, Adult-onset autosomal dominant leukodystrophy (ADLD), Angelman syndrome (Type 1), Angelman syndrome (Type 2), ATR-16 syndrome, AZFa, AZFb, AZFb+AZFc, AZFc, Cat-Eye Syndrome (Type I), Charcot-Marie-Tooth syndrome type 1A (CMT1A), Cri du Chat Syndrome (5p deletion), Early-onset Alzheimer disease with cerebral amyloid angiopathy, Familial Adenomatous Polyposis, Hereditary Liability to Pressure Palsies (HNPP), Leri-Weill dyschondrostosis (LWD)—SHOX deletion, Miller-Dieker syndrome (MDS), NF1-microdeletion syndrome, Pelizaeus-Merzbacher disease, Potocki-Lupski syndrome (17p 11.2 duplication syndrome), Potocki-Shaffer syndrome, Prader-Willi syndrome (Type 1), Prader-Willi Syndrome (Type 2), RCAD (renal cysts and diabetes), Rubinstein-Taybi Syndrome, Smith-Magenis Syndrome, Sotos syndrome, Split hand/foot malformation 1 (SHFM1), Steroid sulphatase deficiency (STS), WAGR 11p 13 deletion syndrome, Williams-Beuren Syndrome (WBS), Wolf-Hirschhorn Syndrome, and Xq28 (MECP2) duplication.

The methods described herein can be used to detect and/or quantitate a DNA epigenetic change including but not limited to chemical modifications and chromatin structure. In some embodiments the DNA epigenetic change comprises a chemical modification. In some embodiments, the chemical modification comprises DNA methylation.

The present invention provides a method for determining methylation status of CpG dinucleotides within a target nucleic acid molecule. CpG islands (a stretch of CpGs), are typically unmethylated. Hypermethylation in CpG islands of promoter regions leads to silence the associated gene expression. Aberrant methylation has been associated to different pathogenesis including neoplasia. In some embodiments, the methods described herein can discriminate and quantitate the methylation state of at least 1, 2, 3, 4, 5, 10, 20, 50, 100, 200, 500, 1,000, 2,000, 5,000, 10,000, 20,000, 50,000, 100,000, different target nucleic acids. In some embodiments, the methods described herein are used to detect and/or quantify methylation status of target nucleic acids with similar sequences. The methods described herein can discriminate and quantitate the methylation state of target nucleic acids varying by 1 nt or more than 1, 2, 3, 4, 5, 10, 12, 15, 20 nt.

In some embodiments, the methods described herein are used to detect and/or quantify gene expression. In some embodiments, the methods described herein provide high discriminative and quantitative analysis of multiples genes. The methods described herein can discriminate and quantitate the expression of at least 1, 2, 3, 4, 5, 10, 20, 50, 100, 200, 500, 1,000, 2,000, 5,000, 10,000, 20,000, 50,000, 100,000, different target nucleic acids.

### Amplification Procedures

The practice of one or more methods disclosed herein may involve amplifying a target nucleic acid and/or a reference nucleic acid. In some embodiments, at least one target nucleic acid and at least one reference nucleic acid are amplified using a different set of primers. That is, a first set of primer specific for the target nucleic acid and a second set of primers specific for a reference nucleic acid are used for the amplification. Typically, the target nucleic acid and the reference nucleic acid are comparable in length.

Usually the quantity of the reference nucleic acid is known. In some embodiments, a reference nucleic acid is added to the sample containing the target nucleic acid. The reference nucleic acid may be isolated from the same or different source as the target nucleic acid. In addition, the reference nucleic acid may be recombinantly produced or artificially synthesized. For example, the target nucleic acid and the reference nucleic acid can be different parts of a single cDNA transcript, such as 5′ end vs. 3′ end. Alternatively, the target nucleic acid and the reference nucleic acid can be different transcripts from a single cDNA preparation. The target nucleic acid and the reference nucleic acid can be different parts from a single gene, or two different regions of the same chromosome, or two different regions from the same human DNA from the same or different sample preparations. Where desired, the reference nucleic acid has a length comparable to that of the target nucleic acid. For example, a reference nucleic acid differs in length as compared to the target nucleic acid by less than about 50%, 40%, 30%, 20%, 10%, 5% or less. A reference nucleic acid typically exhibits a distinct melting profile from that of the target nucleic acid.

Amplification of target nucleic acids can be performed by any means known in the art. In some cases, target nucleic acids are amplified by polymerase chain reaction (PCR). Examples of PCR techniques that can be used include, but are not limited to, quantitative PCR, quantitative fluorescent PCR (QF-PCR), multiplex fluorescent PCR (MF-PCR), real time PCR(RT-PCR), single cell PCR, restriction fragment length polymorphism PCR (PCR-RFLP), PCR-RFLP/RT-PCR-RFLP, hot start PCR, nested PCR, in situ polonony PCR, in situ rolling circle amplification (RCA), bridge PCR, picotiter PCR and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938.

In any of the embodiments, amplification of target nucleic acids and/or reference nucleic acid occurs on a solid support including but not limited to beads, nanoparticles and quantom dots.

In any of the embodiments herein, the nucleic acid(s) of interest can be pre-amplified prior to the amplification step (e.g., PCR). In some cases, a nucleic acid sample may be pre-amplified to increase the overall abundance of genetic material to be analyzed (e.g., DNA). Pre-amplification can therefore include whole genome amplification such as multiple displacement amplification (MDA) or amplifications with outer primers in a nested PCR approach.

In some embodiments, the target nucleic acid is amplified through other isothermal amplification schemes known in the art. In some embodiments of the invention, the target nucleic acid is quantified before the amplification steps.

### Melting Analysis

The methods describe herein generally utilize melting analysis to detect and quantify target nucleic acids. Results from melting analysis with a test sample containing a target nucleic acid of interest are optionally compared with similar target nucleic acids from a control samples, e.g. control cell population. Melting, also called denaturation, is the process by which double-stranded nucleic acid unwinds and separates into single-stranded strands through the breaking of hydrogen bonding between the bases. The melting temperature (Tm) is defined as the temperature at which half of the nucleic acid strands are in the double-helical state and half are in the “random-coil”state. The melting temperature typically depends on both the length of the molecule, and the GC content of that molecule. Short nucleic acid fragments usually have only one melting unit manifested as a single melting peak, while long nucleic acid fragment may have multiple melting units, manifested as multiple melting curves.

Melting analyses can be conviniently done in the presence of a nucleic binding dye concurrent with or after an amplification reaction is completed. When thermal resolution is at 0.05° C. or smaller, melting analyses called high-resolution melting analyses (HRM) are used. HRM could provide very detailed Tm information, including single base changes such as SNPs (single nucleotide polymorphisms).

Nucleic acids from samples that can be analyzed by the methods herein include: double-stranded DNA, single-stranded DNA, single-stranded DNA hairpins, DNA/RNA hybrids, RNA (e.g. mRNA or miRNA) and RNA hairpins.

In some embodiments, to quantify the target nucleic acid content in a target sample, a reference a reference nucleic acid with known quantity is added. The reference and the target nucleic acid can be amplified as described above. The reference nucleic acid may be isolated from the same or different source as the target nucleic acid. In addition, the reference nucleic acid may be recombinantly produced or artificially synthesized. For example, the target nucleic acid and the reference nucleic acid can be different parts of a single cDNA transcript, such as 5′ end vs. 3′ end. Alternatively, the target nucleic acid and the reference nucleic acid can be different transcripts from a single cDNA preparation. The target nucleic acid and the reference nucleic acid can be different parts from a single gene, or two different regions of the same chromosome, or two different regions from the same human DNA from the same or different sample preparations. Typically, the target nucleic acid and the reference nucleic acid are comparable in length, e.g., when the target nucleic acid and reference nucleic are not known to be in similar quantity, or when the target nucleic acid and reference nucleic are known to be in similar quantity. The relative length of the target and reference could be adjusted according to the quantities of the two so that the melting areas are comparable in melting analysis.

In some embodiments, the target nucleic acid analyzed by the methods described herein is a genomic DNA region. In some cases, the genomic DNA region contains one or more polymorphisms such as a CNVR or a STRs or SNPs. The reference nucleic acid could also be a genomic DNA region. Alternatively, the reference nucleic acid could be a cDNA transcript or an oligonucletide. The reference nucleic acid may be isolated from the same or different source as the target nucleic acid. In addition, the reference nucleic acid may be recombinantly produced or artificially synthesized. For example, the target nucleic acid and the reference nucleic acid can be different parts of a single cDNA transcript, such as 5′ end vs. 3′ end. Alternatively, the target nucleic acid and the reference nucleic acid can be different transcripts from a single cDNA preparation. The target nucleic acid and the reference nucleic acid can be different parts from a single gene, or two different regions of the same chromosome, or two different regions from the same human DNA from the same or different sample preparations.

Melting analysis can be carried out using any suitable instrument known in the art such as a UV spectrophotometer. Typical melting analyses can be done on instruments, such as ABI 7900, 7500, BioRad iQ5, Chromo4, Corbett Rotogene 6000, Roche Lightcycler 480, Idaho technology Genetyper, or Stratagene MX4000.

In some embodiments, melting analyses are performed by measuring the absorbance of the target nucleic acid and the reference nucleic acid at a plurality of temperatures. Typically, absorbance is measured at 260.

Melting analysis can also be performed in the presence of a nucleic acid binding agent. In some embodiments, the binding agent emits a signal when the agent is bound to a nucleic acid. The signal can be fluorescent signal, magnetic signal, radioactive signal, Raman signal or an electrochemical signal. In some embodiments the binding agent is a nucleic acid intercalator. Examples of nucleic acid binding agents that can be used in the methods described herein include, but are not limited to, EvaGreen® (Biotium, Hayward, Calif.), SYBR® Green I, PicoGreen™, LC Green™, SYBR GreenER®, PO-PRO®.-1, BO-PRO®.-1, SYTO® 9, SYTO®™43, SYTO®. 44, SYTO®. 45, SYTOX® Blue, POPO™.-1, POPO™.-3, BOBO™.-1, BOBO™-3, LO-PRO™-1, JO-PRO™-1, YO-PRO®-1, TO-PRO®-1, SYTO® 9, SYTO®11, SYTO®13, SYTO®15, SYTO®16, SYTO®20, SYTO®23, TOTO™3, YOYO®-3 (Molecular Probes, Inc., Eugene, Oreg.), GelStar® (Cambrex Bio Science Rockland Inc., Rockland, Me.), Ethidium Bromide, thiazole orange (Aldrich Chemical Co., Milwaukee, Wis.), BEBO, BETO, BOXTO (TATAA Biocenter AB., Goteborg, Sweden).

The binding agent can emit a signal when bound to either single or double stranded nucleic acids. Alternative, the binding agent can emit a signal when is no longer bound to either single or double stranded nucleic acids. In other embodiments, the absorbance of a binding agent is measured to perform the melting analysis described herein. All these type of binding agents can be used and are encompassed in the methods described herein.

The change in the signal from the nucleic acid binding agent (e.g., fluorescence change) or absorbance (e.g. binding agent or nucleic acid) is monitored at different temperatures. For example, in some embodiments, the signal from the nucleic acid binding agent can be monitored at increasing temperatures from 0° C. to 100° C. The intensities of the signals of the binding agent are typically inversely proportional to the degree of melting of a nucleic acid molecule in double stranded or multi-stranded state. The intensity readout at various temperatures points are recorded and plotted to derive a melting curve or a melting profile of the target or reference nucleic acids. Typical melting analyses can be automatically performed on instruments, such as ABI 7900, 7500, BioRad iQ5, Chromo4, Corbett Rotogene 6000, Roche Lightcycler 480, Idaho technology Genetyper, or Stratagene MX4000. Any instrument known in the art suitable for melting analysis can be used with the methods described herein.

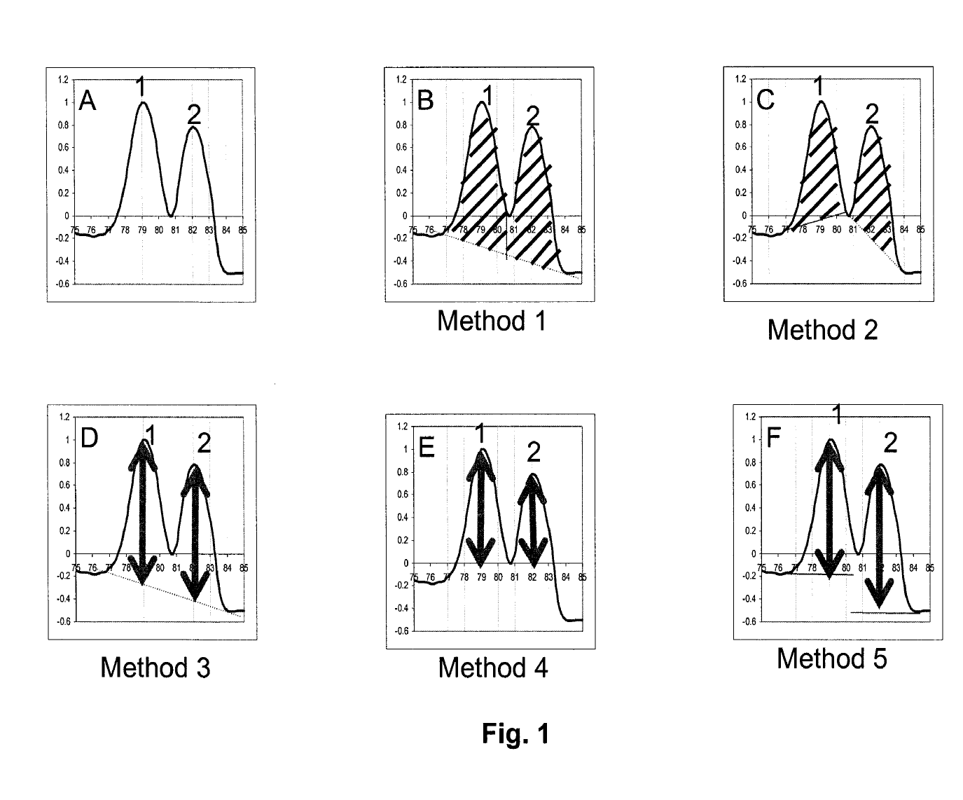

In some embodiments a melting curve is generated from the melting analysis described above. FIG. 1A shows an example of a melting curve obtained after performing melting analyses of the target and reference nucleic acid. The x-axis is the temperature (T), the y-axis is the −dF/dT, where F is the fluorescence intensity recorded. dF/dT is the first derivative against the temperature. Two melting peaks (1 and 2) correspond to reference nucleic acid and target nucleic acid respectively.

There are various ways in which the nucleic acid content can be quantified. In some embodiments, the melting profiles from the target and references nucleic acids are compared to determine the relative amount of signal attributable to the target nucleic acid or the reference nucleic acid based on their melting profile. In some embodiments, the relative amount of the signal attributable to the target nucleic acid and the reference nucleic acid are compared. In some embodiments, a ratio is calculated between the amount of signal attributable to the target nucleic acid and the amount of signal attributable to the reference nucleic acid. The quantity of nucleic acid can be calculated in grams, Dalton, bp, or copy numbers.

FIGS. 1B, 1C, 1D and 1E show various exemplary embodiments for the quantification of nucleic acid content. One method to determine nucleic acid quantity is to measure the area under the peak as shown in FIGS. 1B, and 1C. In general, the amount of target nucleic acid and the area of the melting peak follow the formula I as shown below:

(Peak Area 1)/(Peak Area 2)=(Amount of DNA 1)/(Amount of DNA 2) (I)

In many cases, the area of the peak is proportional to the peak height, the formula I can be modified to formula II as shown below:

(Peak Height 1)/(Peak height 2)=(Amount of DNA 1)/(Amount of DNA 2) (II)

Another method to determine nucleic acid quantity is by measuring the peak height. Three examples are presented in FIGS. 1D, 1E and 1F.

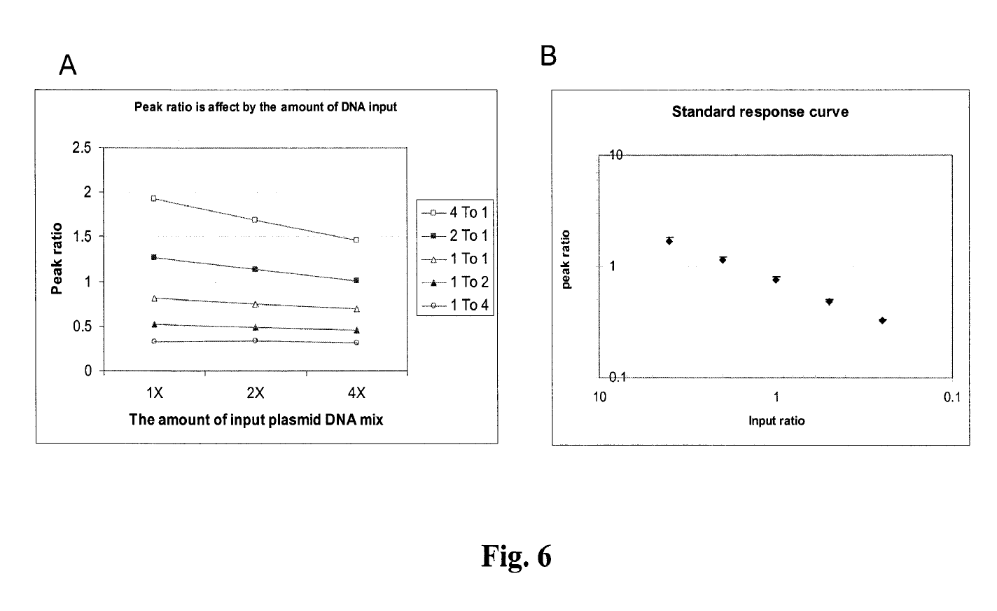

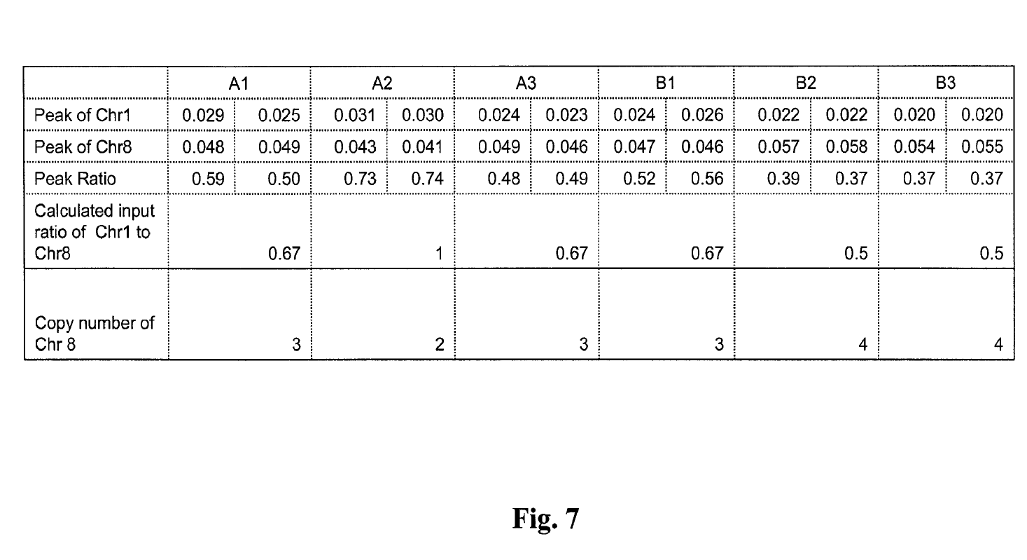

In some embodiments, the quantity of nucleic acid is determined by comparing the results of the melting analyses of the target and reference nucleic acid to a standard curve. The quantity of nucleic acid can be calculated in grams, Dalton, bp, or copy numbers. For instance, after determining the area under the peak or the peak height ratio of the target and reference nucleic acids as described above, the quantity of target nucleic acid can be determined using a standard curve such as the one depicted in FIG. 6B. In Example 7 using the peak-height ratio of the standard response curve in FIG. 6B, the input ratio of Chrl to Chr8 was determined as shown in Row 5 of FIG. 7.

In some embodiments, the methods described herein are used to detect and/or quantified a target nucleic acid molecule. In some embodiments, the methods described herein are used to detect and/or quantified multiple target nucleic acid molecules. The methods described herein can analyzed at least 1, 2, 3, 4, 5, 10, 20, 50, 100, 200, 500, 1,000, 2,000, 5,000, 10,000, 20,000, 50,000, 100,000, different target nucleic acids.

In some embodiments, less than 1 pg, 5 pg, 10 pg, 20 pg, 30 pg, 40 pg, 50 pg, 100 pg, 200 pg, 500 pg, 1 ng, 5 ng, 10 ng, 20 ng, 30 ng, 40 ng, 50 ng, 100 ng, 200 ng, 500 ng, 1 ug, 5 ug, 10 ug, 20 ug, 30 ug, 40 ug, 50 ug, 100 ug, 200 ug, 500 ug or 1 mg of nucleic acids are obtained from the sample for further genetic analysis. In some cases, about 1-5 pg, 5-10 pg, 10-100 pg, 100 pg-1 ng, 1-5 ng, 5-10 ng, 10-100 ng, 100 ng-1 ug of nucleic acids are obtained from the sample for further genetic analysis.

In some embodiments, the methods described herein are used to detect and/or quantified a target nucleic acid molecule which is about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 400, 410, 420, 430, 440, 450, 460, 470, 480, 490, 500, 510, 520, 530, 540, 550, 560, 570, 580, 590, 600, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 by in length. In some embodiments, the methods described herein are used to detect and/or quantified a target nucleic acid molecule which larger that 1 kb in length. In some embodiments, the methods described herein are used to detect and/or quantified a target nucleic acid molecule which is about 1, 2, 3, 4, 5, 6, 7, 8, 9, 10 kb in length.

The methods described herein can be employed to discriminate between the amount of nucleic acid (e.g. copy number) and nucleic acid sequences. The difference between the target nucleotide sequences can be, for example, a single nucleic acid base difference, deletion, insertion, amplification or rearrangement. As a result, the process of the present invention is able to detect infectious diseases, genetic diseases, and cancer. It is also useful in environmental monitoring, forensics, and food science. Examples of genetic analyses that can be performed on nucleic acids include e-g., CNVR detection, SNP detection, STR detection, RNA expression analysis, promoter methylation, gene expression, virus detection, viral subtyping and drug resistance.

A wide variety of infectious diseases can be detected by the process of the present invention. Typically, these are caused by bacterial, viral, parasite, and fungal infectious agents. The resistance of various infectious agents to drugs can also be determined using the present invention.

Bacterial infectious agents which can be detected by the present invention include Escherichia coli, Salmonella, Shigella, Klebsiella, Pseudomonas, Listeria monocytogenes, Mycobacterium tuberculosis, Mycobacterium aviumintracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Acitnomycetes.

Fungal infectious agents which can be detected by the present invention include Cryptococcus neoformans, Blastomyces dermatitidis, Histoplasma capsulatum, Coccidioides immitis, Paracoccidioides brasiliensis, Candida albicans, Aspergillus fumigautus, Phycomycetes (Rhizopus), Sporothrix schenckii, Chromomycosis, and Maduromycosis.

Viral infectious agents which can be detected by the present invention include human immunodeficiency virus, human T-cell lymphocytotrophic virus, hepatitis viruses (e.g., Hepatitis B Virus and Hepatitis C Virus), Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

Parasitic agents which can be detected by the present invention include Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

The present invention is also useful for detection of drug resistance by infectious agents. For example, vancomycin-resistant Enterococcus faecium, methicillin-resistant Staphylococcus aureus, penicillin-resistant Streptococcus pneumoniae, multi-drug resistant Mycobacterium tuberculosis, and AZT-resistant human immunodeficiency virus can all be identified with the present invention.

Genetic diseases can also be detected by the process of the present invention. This can be carried out by prenatal or post-natal screening for chromosomal and genetic aberrations or for genetic diseases. Examples of detectable genetic diseases include: 21 hydroxylase deficiency, cystic fibrosis, Fragile X Syndrome, Turner Syndrome, Duchenne Muscular Dystrophy, Down Syndrome or other trisomies, heart disease, single gene diseases, HLA typing, phenylketonuria, sickle cell anemia, Tay-Sachs Disease, thalassemia, Klinefelter Syndrome, Huntington Disease, autoimmune diseases, lipidosis, obesity defects, hemophilia, inborn errors of metabolism, diabetes, trisomy 13, trisomy 18, trisomy 21, Klinefelter Syndrome, dup (17)(p 11.2p 11.2) syndrome, Down syndrome, Pre-eclampsia, Pre-term labor, Edometriosis, Pelizaeus-Merzbacher disease, dup (22)(q11.2q11.2) syndrome, Cat eye syndrome, Cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Kallmann syndrome, microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c), and 1p 36 deletion.

Cancers which can be detected by the process of the present invention generally involve oncogenes, tumor suppressor genes, or genes involved in DNA amplification, replication, recombination, or repair Examples of these include: BRCA1 gene, p 53 gene, APC gene, Her2/Neu amplification, Bcr/Ab1, K-ras gene, and human papillomavirus Types 16 and 18. Various aspects of the present invention can be used to identify amplifications, large deletions as well as point mutations and small deletions/insertions of the above genes in the following common human cancers: leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, head and neck tumors, and cervical neoplasms. Examples of different cancer and/or neoplastic conditions include but are not limited to acute lymphoblastic leukemia, acute or chronic lymphocyctic or granulocytic tumor, acute myeloid leukemia, acute promyelocytic leukemia, adenocarcinoma, adenoma, adrenal cancer, basal cell carcinoma, bone cancer, brain cancer, breast cancer, bronchi cancer, cervical dysplasia, chronic myelogenous leukemia, colon cancer, epidermoid carcinoma, Ewing's sarcoma, gallbladder cancer, gallstone tumor, giant cell tumor, glioblastoma multiforma, hairy-cell tumor, head cancer, hyperplasia, hyperplastic corneal nerve tumor, in situ carcinoma, intestinal ganglioneuroma, islet cell tumor, Kaposi's sarcoma, kidney cancer, larynx cancer, leiomyomater tumor, liver cancer, lung cancer, lymphomas, malignant carcinoid, malignant hypercalcemia, malignant melanomas, marfanoid habitus tumor, medullary carcinoma, metastatic skin carcinoma, mucosal neuromas, mycosis fungoide, myelodysplastic syndrome, myeloma, neck cancer, neural tissue cancer, neuroblastoma, osteogenic sarcoma, osteosarcoma, ovarian tumor, pancreas cancer, parathyroid cancer, pheochromocytoma, polycythemia vera, primary brain tumor, prostate cancer, rectum cancer, renal cell tumor, retinoblastoma, rhabdomyosarcoma, seminoma, skin cancer, small-cell lung tumor, soft tissue sarcoma, squamous cell carcinoma, stomach cancer, thyroid cancer, topical skin lesion, veticulum cell sarcoma, and Wilm's tumor.

The methods described herein can be used to determine whether an individual is at risk to develop a disease. Examples of such diseases include, but are not limited to HIV infection, glomerulonephritis, CHARGE syndrome, Parkinson's disease and Alzheimer's disease.

In the area of environmental monitoring, the present invention can be used for detection, identification, and monitoring of pathogenic and indigenous microorganisms in natural and engineered ecosystems and microcosms such as in municipal waste water purification systems and water reservoirs or in polluted areas undergoing bioremediation. It is also possible to detect plasmids containing genes that can metabolize xenobiotics, to monitor specific target microorganisms in population dynamic studies, or either to detect, identify, or monitor genetically modified microorganisms in the environment and in industrial plants.

The present invention can also be used in a variety of forensic areas, including for human identification for military personnel and criminal investigation, paternity testing and family relation analysis, HLA compatibility typing, and screening blood, sperm, or transplantation organs for contamination.

In the food and feed industry, the present invention has a wide variety of applications. For example, it can be used for identification and characterization of production organisms such as yeast for production of beer, wine, cheese, yogurt, bread, etc. Another area of use is with regard to quality control and certification of products and processes (e.g., livestock, pasteurization, and meat processing) for contaminants. Other uses include the characterization of plants, bulbs, and seeds for breeding purposes, identification of the presence of plant-specific pathogens, and detection and identification of veterinary infections.

### Instruments

Also provided herein is an instrument for use in a melting analysis described herein comprising multiple thermal cycles, comprising: an automated thermal cycler capable of alternately heating and cooling, and adapted to receive, at least one reaction vessel containing an reaction mixture comprising a target nucleic acid, a reference nucleic agent, and nucleic acid binding agent; wherein the cycler is programmable to control temperature. The reaction mixture may also comprise reagents to perform an amplification reaction. In some embodiments, the instrument additionally comprises a display capable of indicating the melting profile of the target and reference nucleic acid. Such a display may aid the user of the instrument in performing the methods disclosed herein.

The instrument may further comprise a detector operable to detect a fluorescence optical signal while the melting analysis is in progress. The detector is for example operable to detect a fluorescence optical signal in at least one of the following wavelength regions: from about 510 to about 530 nm, from about 540 to about 550 nm, from 560 to about 580 nm, from about 585 to about 595 nm, from 590 to about 610 nm, from 660 to about 680 nm, from about 690 to about 710 nm, or from 770 to about 790 nm. The instrument may also be adapted to receive a plurality of reaction vessels, each containing an reaction mixture.

Other instruments known in the art are also suitable for performing the methods of the invention. Such instruments are described, for example, in U.S. Pat. Nos. 6,814,934, 5,475,610, 5,928,907, 5,972,716, and 6,015,674, all of which are hereby incorporated by reference.

Also provided herein are algorithms and computer software programs capable of analyzing melting analysis as described herein to determine the amount of one or more target nucleic acids. The quantity of nucleic acid can be calculated in grams, Dalton, bp, or copy numbers. Such algorithms and computer software programs may aid the user of the instrument in performing the methods disclosed herein. This algorithms and computer software programs can be attached, incorporated or separate to the instrument running the melting analysis. The quantity of the target nucleic acid can be determine instantly as the instrument is running the analysis or it can be calculated at a later point.

### Kits

In an embodiment, a kit is provided for a detection and/or quantitation of a target nucleic acid. The kit includes: a nucleic acid binding agents as described herein and an oligo mix containing the oligonucleotide probes described herein. In addition, kits are provided which comprise reagents and instructions for performing methods of the present invention, or for performing tests or assays utilizing any of the compositions, or assemblies of articles of the present invention. The kits may further comprise buffers, restriction enzymes, adaptors, primers, a polymerase, dNTPS, NTPs, detection reagents and instructions necessary for use of the kits, optionally including troubleshooting information.

### EXAMPLES

### Example 1

### Preparation of a Standard of Reference DNA

In a 20 μL reaction volume, a 10 ng fragment of DNA, which resides in human Chromosome 1, containing the sequence 5′-TGATTCTCTATACCCATTATGACCTGGATATTGGTATTATTGTGGCCATTTCTACCTCAT CACACGTTCTGGAGAATTGT-3′ (SEQ ID NO: 1) was amplified using primers, ChrlF (5′-TTGATTCTCTATACCCATT-3′, SEQ ID NO: 2) and ChrlR (5′-AACAATTCTCCAGAACGTG-3′, SEQ ID NO: 3), in the presence of 1× of EvaGreen® qPCR Basic Mix (Biotium, Hayward, Calif.) and 1 unit of Taq polymerase (Fermentas). The following thermocyle procedure was used for amplification: 95° C. for 4 minutes, 40 cycles of 95° C. for 15 second, 45° C. for 60 second, and 60° C. for 60 second. Standard agarose gel electrophoresis was used to confirm amplification of the DNA fragment. Using standard cloning protocols, the resulting amplified DNA fragment was cloned into pTOPO CRII vector (Invitrogen, Carlsbad, Calif.) to generate the pChrl plasmid.

### Example 2

### Preparation of a Standard of Target DNA

In a 20 μL reaction volume, al0 ng fragment of DNA, which resides in human Chromosome 8, containing the sequence 5′-ATTTAAACGGATAGTTCTGCAGCCTGAACTTAAATGTTTTCAGGATAAAACAGTTTCAAA

AATGACTTACCGAAAATCTTCAACTTGTGGCAATGGAATTTTGGAACCTACAGAGCAGTGTGA TTGTGGCTATAAAGA-3′, SEQ ID NO: 4) was amplified using primers, Chr8F (5′-ATTTAAACGGATAGTTCTG-3′, SEQ ID NO: 5) and Chr8R (5′-TCTTTATAGCCACAATCAC-3′, SEQ ID NO: 6) in the presence of 1× of EvaGreen® qPCR Basic Mix (Biotium, Hayward, Calif.) and 1 unit of Taq polymerase (Fermentas). The following thermocyle procedure was used for amplification: 95° C. for 4 minutes, 40 cycles of 95° C. for 15 second, 45° C. for 60 second, and 60° C. for 60 second. Standard agarose gel electrophoresis was used to confirm amplification of the DNA fragment. Using standard cloning protocols, the resulting amplified DNA fragment was cloned into pTOPO CRII vector (Invitrogen, Carlsbad, Calif.) to generate the pChr8 plasmid

### Example 3

### Copy Number Determination of Target DNA Using Real-Time PCR

DNA samples from two sets of family trios were purchased from Coriell Cell Repositories (Salt lake city, Utah): Ref NA10846, Ref NAl2144, Ref NA 12145, Ref NA06994, Ref NA07000, Ref NA07029 (see Redon et al, Nature 444:444-454, 2006) and herein denoted as A1, A2, A3, B1, B2 and B3, respectively. Redon et al determined that these sample DNA, A1, A2, A3, B1, B2, and B3 respectively have 3, 2, 3, 3, 4, and 4 copies in a region of Chromosome 8. Target DNA (SEQ ID NO: 4) from example 2 is within the region of chromosome 8 that exhibits copy number variation.

Each of the six DNA samples (A1, A2, A3, B1, B2, B3) were amplified using 3 sets of primers in 3 separate amplication reactions. All reactions were performed in duplicate. In a 20 μl, reaction volume, al0 ng concentration of each DNA were amplified using 10 μL of 2× 1× of EvaGreen® qPCR Basic Mix HS (Biotium, Hayward, Calif.). For each reaction, 500 nM of one of the following 3 primer pairs were used: (1) ChrlF and ChrlR; (2) Chr8F and Chr8R; and (3) AmelF (5′-CCTGGGCTCTGTAAAGAATAGT-3′, SEQ ID NO: 7) and AmelR (5′-CAGAGCTTAAACTGGGAAGCT-3′, SEQ ID NO: 8). Primers ChrlF and ChrlR were used for Ct determination of chromosome 1. Primers Chr8F and Chr8R were used for Ct determination of chromosome 8. Primers AmelF and AmelR were used for Ct determination of sex chromosomes. The following thermocyle procedure was used for amplification of each reaction: 95° C. for 4 minutes, 30 cycles of 95° C. for 15 second, 45° C. for 60 second, and 60° C. for 60 second.

Ct_{Amel, X} is the Ct generated using the primer pair, AmelF and AmelR, with sample X wherein X is A1, A2, A3, B1, B2, or B3.

Ct_{Chrl, x} is the Ct generated using the primer pair, ChrlF and ChrlR, with sample X wherein X is A1, A2, A3, B1, B2, or B3.

Ct_{Chr8, X} is the Ct generated using the primer pair Chr8F and Chr8R with sample X wherein X is A1, A2, A3, B1, B2, or B3.

ΔΔCt were generated using the following equations III-VI.

The relative Ct difference between the fragment from chromosome 1 and the Amel fragment was obtained using Equation 3:

ΔCt_{Chrl, X}=Ct_{Amel, X}−Ct_{Chrl, X} (III)

The smallest number of ΔCt_{Chrl, X} of the six DNA samples is used as Min(ΔCt_{Chrl, X}).

The relative Ct difference between the fragment from chromosome 8 and the Amel fragment was obtained using Equation 4:

ΔCt_{Chr8, X}=Ct_{Amel, X}−Ct_{Chr8, X} (IV)

The smallest number of ΔCt_{Chr8, X} of the six DNA samples is used as Min(ΔCt_{Chr8, X}).

ΔΔCt_{Chrl, X} is obtained using Equation 5:

ΔΔCt_{Chrl, X}=ΔCt_{Chrl, X}−Min(ΔCt_{Chrl, X} (V);

ΔΔCt_{Chr8, X} is obtained using Equation 6:

ΔΔCt_{Chr8, X}=ΔCt_{Chr8, X}−Min(ΔCt_{Chr8, X} (VI);

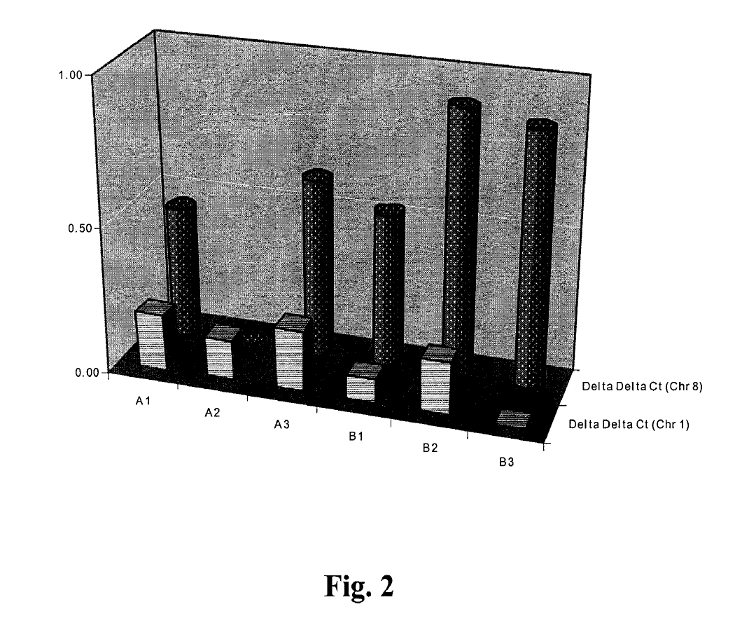

FIG. 2 is a chart plotting copy number determination using ΔΔCt from the real-time PCR assay as described above. A1, A2, A3, B1, B2 and B3 are DNA samples from two family trios as described. According to Redon et al, each DNA sample have exactly 2 copies in a region of chromosome 1, and each respectively have 3, 2, 3, 3, 4, and 4 copies in a region of chromosome 8.

The chart shows ΔΔCt_{Chrl, X} (labeled as Delta Delta Ct (Chrl) and represented by the square columns), and ΔΔCt_{Chrl, X}, (labeled as Delta Delta Ct (Chr8) and represented by the round columns) are plotted following mathematical manipulation as described above. Assuming that each individual has one pair of sex chromosome, each individual must have two copies of the Amel gene. The relative Ct difference between the fragment from chromosome 1 and the Amel fragment and between the fragment from chromosome 8 and Amel fragment were obtained using Equations 3 and 4, respectively and were used to calculate copies of chromosome 1 and 8. After rounding to the nearest digit, DNA samples A1, A2, A3, B1, B2, and B3 were determined to each have 2 copies of chromosome 1 (SEQ ID NO: 1) and 3, 2, 3, 3, 4, and 4 copies of chromosome 8 (SEQ ID NO: 4), respectively. These copy numbers are in agreement with previously published results by Redon et al.

### Example 4

### Standard Response Curve for Calculation of DNA Mix Ratios Using Peak-Height Ratio

In separate reaction tubes, pChrl and pChr8 were mixed in various quantities according to the chart shown below:

| | Mix ratio of pChr1 to pChr8 | | | | | | | | | | |
|---|---|---|---|---|---|---|---|---|---|---|---|
| | 10 TO 1 | 8 TO 1 | 6 TO 1 | 4 TO 1 | 2 TO 1 | 1 TO 1 | 1 TO 2 | 1 TO 4 | 1 TO 6 | 1 TO 8 | 1 TO 10 |
| Units of pChr1 | 10 | 8 | 6 | 4 | 2 | 1 | 1 | 1 | 1 | 1 | 1 |
| Units of pChr8 | 1 | 1 | 1 | 1 | 1 | 1 | 2 | 4 | 6 | 8 | 10 |

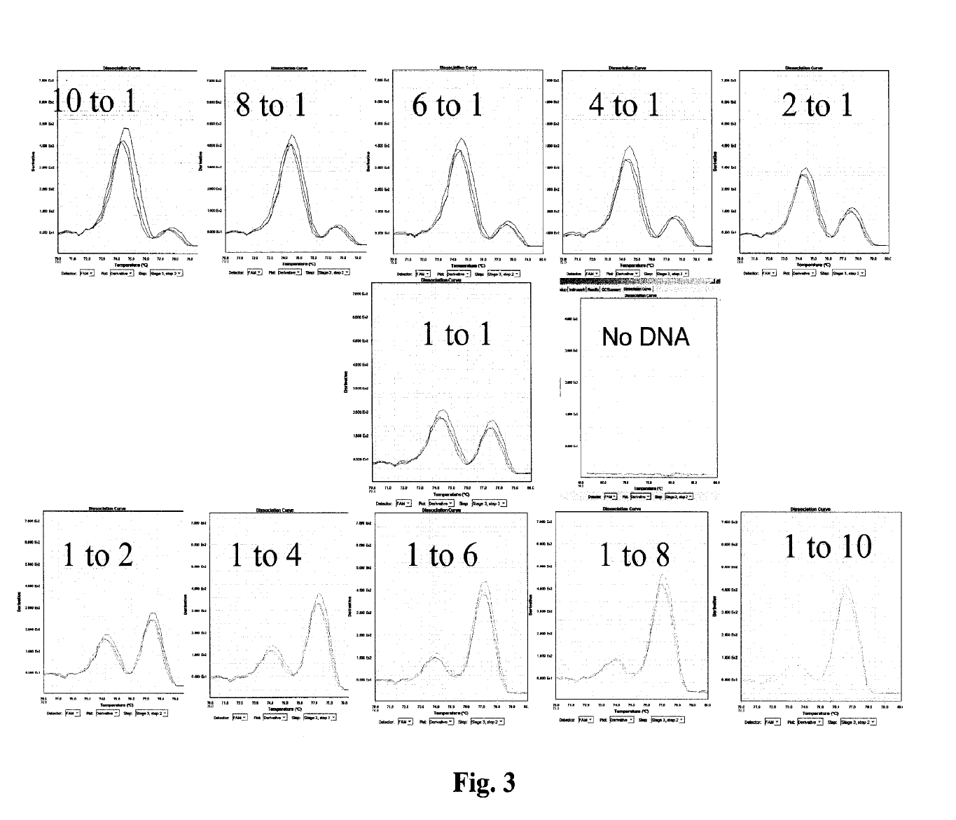

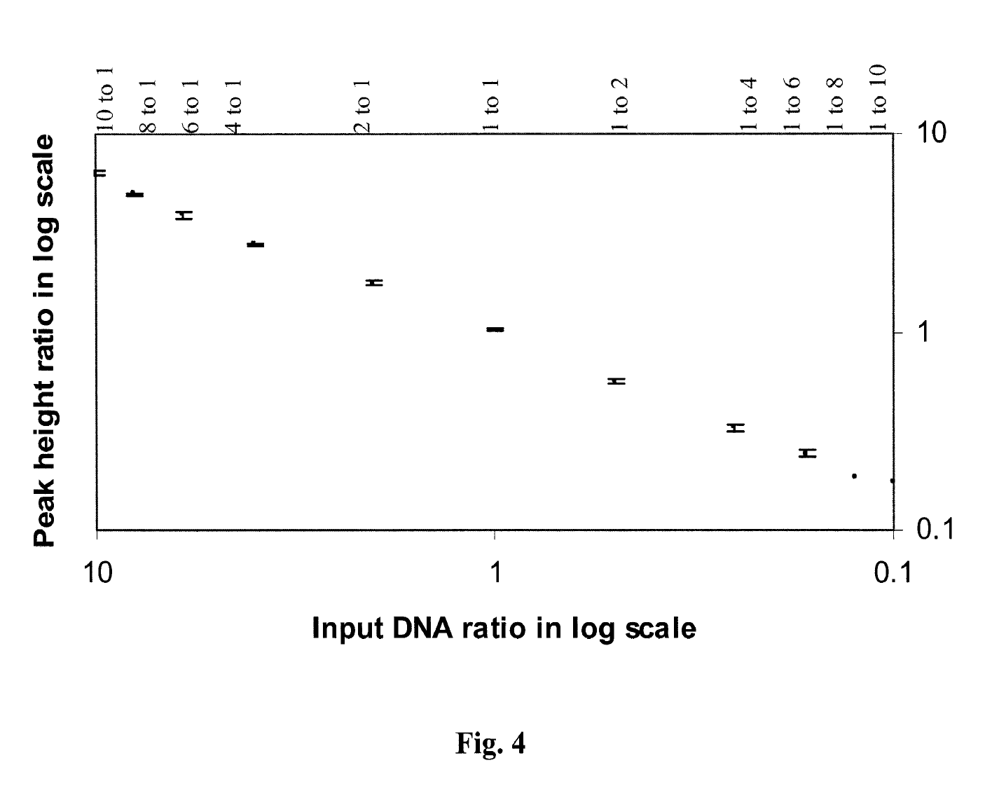

Each unit of pChrl or pChr8 has about 3000 copies of the plasmid per 1 μL. Each reaction containing one of the above DNA mix ratio were amplified in a 20 μL reaction volume using 1 tit of the DNA mix and lx of EvaGreen® qPCR Basic Mix HS (Biotium, Hayward, Calif.) master mix with 2 sets of primers ChrlF, Chrl R, Chr8F and Chr8R in one reaction. The following thermocyle procedure was used for amplification of each reaction: 95° C. for 4 minutes, 30 cycles of 95° C. for 15 second, 45° C. for 60 second, and 60° C. for 60 second. Following amplification, melting analysis was performed in the presence of a DNA binding dye, EvaGreen, from a temperature of 60° C. to 95° C., with increment of 0.2° C. per step and exciting at wavelength of around 488 nm and colleting around 510 nm on a BioRad's iCycler IQ. Other non-limiting examples of DNA binding dyes include SYBR Green I, PicoGreen, Cyto 9, LC Green, SYBR GreenER, Ethidium Bromide, TOTO, YOYO, Bebo, and Bexto. Non-limiting examples of various instruments that can be used to perform melting analyses are ABI 7900, 7500, BioRad iQS, Chromo4, Corbett Rotogene 6000, Roche Lightcycler 480, Idaho technology Genetyper, or Stratagene MX4000. FIG. 3 shows the melting curves generated from the melting analyses. The peak-height ratio was calculated using panel 5 of FIG. 1 and plotted against the DNA mix ratio for each reaction in the above chart (FIG. 4). Results from FIG. 4 indicate that the peak-height ratio is linearly correlated to the input DNA mix ratio. The linear correlation demonstrates that calculation of the peak-height ratio allows the input DNA mix ratio to be determined.

### Example 5

### Annealing Time Affects the Peak-Height Ratios but does not Affect not the Linearity of the Peak-Height Ratios to the Input DNA Mix Ratios

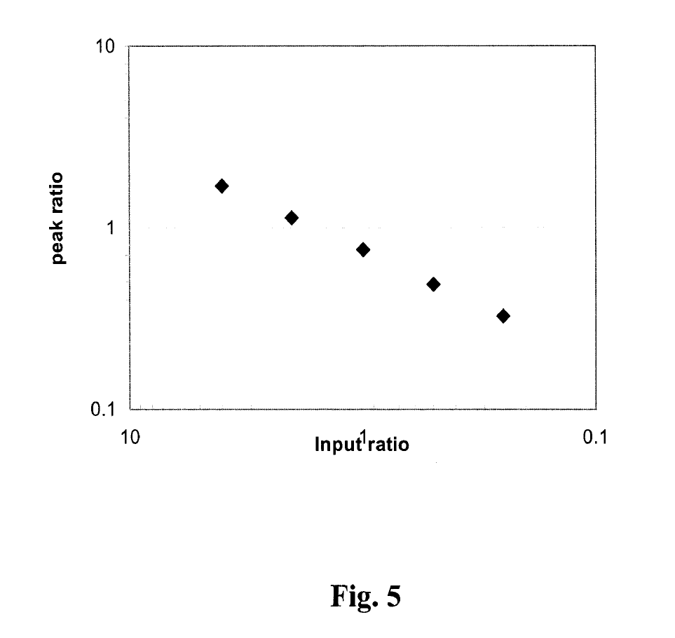

Five DNA mix ratios were prepared (units of pChrl to units of pChr8): 4 to 1, 2 to 1, 1 to 1, 1 to 2, and 1 to 4. Each unit of pChrl or pChr8 has about 3000 copies of the plasmid per 1 μL. Each reaction containing one of the above DNA mix ratio were amplified in a 20 μL reaction volume using 1 μL of the DNA mix and 1× of EvaGreen® qPCR Basic Mix HS (Biotium, Hayward, Calif.) with 2 sets of primers ChrlF, ChrlR, Chr8F and Chr8R in one reaction. The following thermocyle procedure was used for amplification of each reaction: 95° C. for 4 minutes, 30 cycles of 95° C. for 15 second, 45° C. for 90 second, and 60° C. for 60 second. Following amplification, melting analysis was performed and the melting curves were generated as described (not shown). The peak-height ratio was calculated using panel 5 of FIG. 1 and plotted against the DNA mix ratio for each reaction in the above chart (FIG. 5). Results from FIG. 5 indicate that a different annealing time (90 s) from the annealing time used in Example 4 (60 s) during amplification does not affect the linearity of the peak-height ratios to the input DNA mix ratios. However the different annealing time used resulted in differences of the absolute numbers of the peak-height ratio. Results show that a strict thermocycle procedure should be followed during amplification. In addition, we observed different brand of instrument may yield different absolute melting profiles. Different manufactures use different temperature increment to collect melting fluorescence, and may use different mathematic algorithm for data treatment as their default settings. The absolute melting profiles of the same sample may look different from instruments of different manufacturers; however, results are quite repeatable when the melting was performed on the same instrument.

### Example 6

### Starting Quantity of Input DNA Mix Amount Affects the Peak Ratio but does not Affect not the Linearity of the Peak-Height Ratios to the Input DNA Mix Ratios

Five DNA mix ratios were prepared (units of pChrl to units of pChr8): 4 to 1, 2 to 1, 1 to 1, 1 to 2, and 1 to 4. For each DNA mix ratio in a 20 μL reaction volume, 1 μL, 2 μL, and 4 μl, (labeled as 1×, 2×, and 4×, respectively, in FIG. 6A) of DNA mix ratio were separately amplified using 1× of EvaGreen® qPCR Basic Mix HS (Biotium, Hayward, Calif.) with 2 sets of primers ChrlF/ChrlR and Chr8F/Chr8R in one reaction. The following thermocyle procedure was used for amplification of each reaction: 95° C. for 4 minutes, 30 cycles of 95° C. for 15 second, 45° C. for 90 second, and 60° C. for 60 second. Following amplification, melting analysis was performed and the melting curves were generated as described (not shown). The peak-height ratio was calculated using panel 5 of FIG. 1 and plotted against the starting quantity of input DNA (1×, 2×, 4×) for each reaction in the above chart (FIG. 6A). Results from FIG. 6A indicate that a different starting quantity of input DNA affects the peak-height ratios. As the starting quantity of input DNA increased, the peak-height ratio decreased. In addition, the extent to which the peak-height ratio was affected is a function of the input DNA mix ratio; the degree of decrease in the peak-height ratio became less noticeable as the relative amount of pChrl to pChr8 decreased.

Although the starting quantity of input DNA affects the peak-height ration, the input DNA amount can be calculated by the Ct and therefore the ratio corresponding to a fixed amount of input DNA can be corrected. FIG. 6B shows a representative standard response curve with peak-height ratio vs. input DNA mix ratio using the means of peak-height ratios of lx, 2×, and 4× starting amounts. Results show a linear correlation and that each point is well separated. A plot of the Ct corrected ratio vs. input DNA mix ratio showed that the error bars of the points are small for the various input amounts. This illustrates that the reaction tolerates at least 4 fold fluctuations in the starting quantity of input DNA.

### Example 7

### Determination of Copy Number of Genomic DNA

Each of the six DNA samples (A1, A2, A3, B1, B2, B3) were amplified using both sets of ChrlF/ChrlR and Chr8F/Chr8R primers together. All reactions were performed in duplicate. In a 20 μL reaction volume, a 10 ng concentration of each DNA were amplified using 1× of EvaGreen® qPCR Basic Mix HS (Biotium, Hayward, Calif.), Taq polymerase, and both sets of primers. The following thermocyle procedure was used for amplification of each reaction: 95° C. for 4 minutes, 30 cycles of 95° C. for 15 second, 45° C. for 60 second, and 60° C. for 60 second.

Following amplification, melting analysis was performed and the melting curves were generated as described (not shown). The peak-height ratio of Chrl and Chr8 were calculated using panel 5 of FIG. 1 and are shown in FIG. 7, rows 2 and 3, respectively. Row 4 of FIG. 7 shows the peak-height ratio of Chrl to Chr8. Using the peak-height ratio of the standard response curve in FIG. 6B, the input ratio of Chrl to Chr8 was determined and shown in Row 5 of FIG. 7. The copy numbers of Chrl (reference DNA) for A1, A2, A3, B1, B2, B3 were previously determined to all be 2 (see Example 3). Using the ratio of Chrl to Chr8 and the copy number of Chrl, the copy number of Chr8 was determined for A1, A2, A3, B1, B2, B3 and are shown in Row 6 to be 3, 2, 3, 3, 4, 4, respectively. These copy number results indicate that the methods disclosed herein are in agreement with the copy number results obtained by the conventional ΔΔCt method (see Example 3) and with the reported numbers of Redon et al.

### Example 8

### Detection of Gene Duplication in Charcot-Marie-Tooth Disease Type 1

Charcot-Marie-Tooth disease type 1A (CMT1A) and hereditary neuropathy with liability to pressure palsies (HNPP) are autosomal dominant disorders associated with DNA duplication or deletion of a specific 1.5-Mb genomic fragment of the peripheral myelin protein 22 (PMP22) gene located at chromosome 17p 11.2-p 12. DNA from individuals with CMT1A or HNPP are extracted from peripheral blood leukocytes using standard methods (see e.g. Sambrook, J. and D. W. Russell (2001). Control DNA samples are obtained from individuals without CMT1A and HNPP to ascertain the absence of a duplication or deletion at 17p 11.2-p 12.

Oligonucleotide primers are designed to amplify part of the PMP22 target sequence that lie within the potentially duplicated or deleted target region and have a different melting profile than a reference region. The Amel gene can be used as the reference gene.

Each DNA sample from CMT1 A, HNPP, and unaffected individuals is amplified using both sets of primer pairs, to amplify the target region (PMP22) and reference region, respectively. All reactions are performed in duplicate. In a 20 uL reaction volume, a10 ng concentration of each DNA is amplified using 1× of EvaGreen® qPCR Basic Mix HS (Biotium, Hayward, Calif.), Taq polymerase, and both sets of primers. The following thermocyle procedure is used for amplification of each reaction: 95° C. for 5 minutes, 26 cycles of 94° C. for 30 second, 58° C. for 30 second, and 72° C. for 30 second. The PCR cycle ends with 72° C. for 10 min.

Following amplification, melting analysis is performed and the melting curves are generated using methods described. The peak-height ratio of the reference region and PMP22 are calculated using the method shown in panel 5 of FIG. 1. A standard response curve is generated using similar procedures as those used to generate FIG. 6B. Using the peak-height ratio of the standard response curve, the input ratio of reference region to PMP22 is determined and allows the amount of product amplified for the PMP22 sequence to be compared with the amount of PCR product generated from the reference region The copy number of the reference is usually known. The copy number of the reference Amel is 2. Using the input ratio of reference region to PMP22 and the known copy number of the reference region, the copy number of PMP22 is determined. The copy number of PMP22 from unaffected individuals is expected to be 2.

### Example 9

### Detection of Amplification of the CTSZ and CD24 Genes in Human Cancers

The Cathepsin Z (CTSZ) and small cell lung carcinoma cluster 4 antigen (CD24) genes are frequently amplified in tumor tissue and cell lines. The human CTSZ gene maps to chromosome 20q13 and CD24 gene is located on human chromosome 6q21. Genomic DNAs are isolated from colon cancer, breast cancer, or ovarian cancer samples using standard protocols known in the art. The Amel gene can be used as the reference gene. Using known protocols in the art, amplification primers directed to the CTSZ or CD24 gene can be designed and generated.

A DNA sample from each type of cancer is amplified using two sets of primer pairs, one directed to amplifying the target gene (CTSZ or CD24) and another directed to amplifying a reference region (e.g. Amel). All reactions are performed in duplicate. In a 20 μL it reaction volume, a 10 ng concentration of each DNA is amplified using 1× of EvaGreen® qPCR Basic Mix HS (Biotium, Hayward, Calif.), Taq polymerase, and both sets of primers. The following thermocyle procedure is used for amplification of each reaction: 95° C. for 5 minutes, 26 cycles of 94° C. for 30 second, 58° C. for 30 second, and 72° C. for 30 second. The PCR cycle ends with 72° C. for 10 min. It is understood that variations in the thermocycle procedure used will depend on various factors such as the size and nucleotide content of the template DNA and primers.

Following amplification, melting analysis is performed and the melting curves are generated using methods described. The peak-height ratio of reference region and either CTSZ or CD24 are calculated using the method shown in panel 5 of FIG. 1. A standard response curve is generated using similar procedures as those used to generate FIG. 6B. Using the peak-height ratio of the standard response curve, the input ratio of reference region to either CTSZ or CD24 is determined and allows the amount of product amplified for either the CTSZ or CD24 sequence to be compared with the amount of PCR product generated from reference region sequence. The copy number of the reference region is usually known. The copy number of the reference Amel is 2. Using the input ratio of reference region to either CTSZ or CD24 and the known copy number of Amel, the copy number of either CTSZ or CD24 is determined. Comparison of the number of CTSZ or CD24 DNA copies detected in the sample to a control or a known value, allows determination of whether the CTSZ or CD24 gene is amplified in the biological test subject. Amplification of the CTSZ or CD24 gene indicates a cancer in the tissue.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for determining the amount of a target nucleic acid comprising:
providing a sample comprising a nucleic acid binding agent, a target nucleic acid and a reference nucleic acid; wherein said target nucleic and said reference nucleic acid exhibit distinct melting profiles and wherein said binding agent yields a detectable signal when bound to the target nucleic acid and/or the reference nucleic acid;
determining the melting profiles of said target nucleic and said reference nucleic acid by detecting the signal of said binding agent at a plurality of temperatures;
comparing the melting profile of said target nucleic acid to the melting profile of said reference nucleic acid; and
determining the amount of said target nucleic acid based on said comparison.

2. The method of claim 1 wherein said target nucleic acid and/or said reference nucleic are amplified products of said target and said reference nucleic acids.

3. The method of claim 2 wherein said amplified products of said target nucleic acid and/or said reference nucleic are amplified by a PCR reaction.

4. The method of claim 2 wherein said amplified products of the target nucleic acid amplified product and said amplified products of the reference nucleic are generated with the use of distinct sets of primers.

5. The method of claim 1 wherein said target nucleic acid comprises a genomic DNA region.

6. The method of claim 5 wherein said region of genomic DNA comprises one or more polymorphisms.

7. The method of claim 5 wherein said region of genomic DNA comprises one or more copy number variable region (CNVR).

8. The method of claim 6 wherein said polymorphisms are STRs or SNPs.

9. The method of claim 1 wherein said target nucleic acid and said reference nucleic acid are double stranded.

10. The method of claim 1 wherein the size of said target nucleic acid is about 100 by to about 1 kilobase.

11. The method of claim 10 wherein said target nucleic acid and said reference nucleic acid are comparable in length.

12. The method of claim 1 wherein said reference nucleic acid comprises a genomic DNA region.

13. The method of claim 1 wherein said reference nucleic acid is a cDNA transcript, or an oligonucleotide.

14. The method of claim 12 wherein the copy number of said reference nucleic acid is known.

15. The method of claim 1 wherein said target nucleic acid is associated with condition.

16. The method of claim 15 wherein said condition is condition is selected from the group consisting of trisomy 13, trisomy 18, trisomy 21, Klinefelter Syndrome, dup (17)(p 11.2p 11.2) syndrome, Down syndrome, Pre-eclampsia, Pre-term labor, Edometriosis, Pelizaeus-Merzbacher disease, dup (22)(q11.2q11.2) syndrome, Cat eye syndrome, Cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Kallmann syndrome, microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c), 1p 36 deletion, and a combination thereof.

17. The method of claim 15 wherein said condition is selected from the group consisting of condition is acute lymphoblastic leukemia, acute or chronic lymphocyctic or granulocytic tumor, acute myeloid leukemia, acute promyelocytic leukemia, adenocarcinoma, adenoma, adrenal cancer, basal cell carcinoma, bone cancer, brain cancer, breast cancer, bronchi cancer, cervical dysplasia, chronic myelogenous leukemia, colon cancer, epidermoid carcinoma, Ewing's sarcoma, gallbladder cancer, gallstone tumor, giant cell tumor, glioblastoma multiforma, hairy-cell tumor, head cancer, hyperplasia, hyperplastic corneal nerve tumor, in situ carcinoma, intestinal ganglioneuroma, islet cell tumor, Kaposi's sarcoma, kidney cancer, larynx cancer, leiomyomater tumor, liver cancer, lung cancer, lymphomas, malignant carcinoid, malignant hypercalcemia, malignant melanomas, marfanoid habitus tumor, medullary carcinoma, metastatic skin carcinoma, mucosal neuromas, mycosis fungoide, myelodysplastic syndrome, myeloma, neck cancer, neural tissue cancer, neuroblastoma, osteogenic sarcoma, osteosarcoma, ovarian tumor, pancreas cancer, parathyroid cancer, pheochromocytoma, polycythemia vera, primary brain tumor, prostate cancer, rectum cancer, renal cell tumor, retinoblastoma, rhabdomyosarcoma, seminoma, skin cancer, small-cell lung tumor, soft tissue sarcoma, squamous cell carcinoma, stomach cancer, thyroid cancer, topical skin lesion, veticulum cell sarcoma, and Wilm's tumor.

18. The method of claim 15 wherein said condition is a risk to develop a disease.

19. The method of claim 18 wherein said disease is selected from the group consisting of HIV infection, glomerulonephritis, CHARGE syndrome, Parkinson's disease and Alzheimer's disease.

20. The method of claim 1 wherein determining the amount of said target nucleic acid further comprises determining the copy number of said target nucleic acid.

21. The method of claim 1 wherein said nucleic acid binding agent is a DNA binding agent.

22. The method of claim 21 wherein said DNA binding agent is a DNA intercalator.

23. The method of claim 21 wherein said DNA binding agent is selected from the group consisting of EvaGreen, SYBR Green I, PicoGreen, Cyto 9, LC Green, SYBR GreenER, Ethidium bromide, TOTO, YOYO, Bebo, SYTO Green and bexto.

24. The method of claim 23 wherein said DNA binding agent is EvaGreen.

25. The method of claim 1 wherein comparing the melting profiles from said target and references nucleic acid comprises determining the relative amount of signal attributable to said target nucleic acid or to said reference nucleic acid based on their melting profile.

26. The method of claim 25 further comprising comparing the relative amount of the signal attributable to said target nucleic acid and said reference nucleic acid.

27. The method of claim 25 further comprising calculating the ratio between the amount of signal attributable to said target nucleic acid and the amount of signal attributable to said reference nucleic acid.

28. The method of claim 1 wherein said detectable signal is a fluorescent signal, magnetic signal, radioactive signal, Raman signal or an electrochemical signal.

29. A method of determining a genetic condition in a patient or a fetus comprising:
providing a sample suspected to contain a target nucleic acid
providing a reference nucleic acid and a nucleic acid binding agent, wherein said target nucleic and said reference nucleic acid exhibit distinct melting profiles;
amplifying said target nucleic acid and said reference nucleic acid using a first set of primers specific for said target nucleic acid and a second set of primes specific from said reference nucleic acid;
determining the melting profiles of said amplified target nucleic acid and said amplified reference nucleic acid by monitoring a signal from said binding agent at a plurality of temperatures;
comparing the melting profile of said target nucleic acid to the melting profile of said reference nucleic acid; and
determining the presence of absence of said genetic condition based on said comparison.

30. The method of claim 29 wherein said condition is condition is selected from the group consisting of trisomy 13, trisomy 18, trisomy 21, Klinefelter Syndrome, dup (17)(p 11.2p 11.2) syndrome, Down syndrome, Pre-eclampsia, Pre-term labor, Edometriosis, Pelizaeus-Merzbacher disease, dup (22)(q11.2q11.2) syndrome, Cat eye syndrome, Cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Kallmann syndrome, microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c), 1p 36 deletion, and a combination thereof.

31. The method of claim 29 wherein said condition is selected from the group consisting of condition is acute lymphoblastic leukemia, acute or chronic lymphocyctic or granulocytic tumor, acute myeloid leukemia, acute promyelocytic leukemia, adenocarcinoma, adenoma, adrenal cancer, basal cell carcinoma, bone cancer, brain cancer, breast cancer, bronchi cancer, cervical dysplasia, chronic myelogenous leukemia, colon cancer, epidermoid carcinoma, Ewing's sarcoma, gallbladder cancer, gallstone tumor, giant cell tumor, glioblastoma multiforma, hairy-cell tumor, head cancer, hyperplasia, hyperplastic corneal nerve tumor, in situ carcinoma, intestinal ganglioneuroma, islet cell tumor, Kaposi's sarcoma, kidney cancer, larynx cancer, leiomyomater tumor, liver cancer, lung cancer, lymphomas, malignant carcinoid, malignant hypercalcemia, malignant melanomas, marfanoid habitus tumor, medullary carcinoma, metastatic skin carcinoma, mucosal neuromas, mycosis fungoide, myelodysplastic syndrome, myeloma, neck cancer, neural tissue cancer, neuroblastoma, osteogenic sarcoma, osteosarcoma, ovarian tumor, pancreas cancer, parathyroid cancer, pheochromocytoma, polycythemia vera, primary brain tumor, prostate cancer, rectum cancer, renal cell tumor, retinoblastoma, rhabdomyosarcoma, seminoma, skin cancer, small-cell lung tumor, soft tissue sarcoma, squamous cell carcinoma, stomach cancer, thyroid cancer, topical skin lesion, veticulum cell sarcoma, and Wilm's tumor.

32. The method of claim 29 wherein said condition is a risk to develop a disease.

33. The method of claim 32 wherein said disease is selected from the group consisting of HIV infection, glomerulonephritis, CHARGE syndrome, Parkinson's disease and Alzheimer's disease.

34. The method of claim 29 wherein said target nucleic acid comprises a genomic DNA region.

35. The method of claim 34 wherein said region of genomic DNA comprises one or more polymorphisms.

36. The method of claim 34 wherein said region of genomic DNA comprises one or more CNVR.

37. The method of claim 35 wherein said polymorphisms are STRs or SNPs.

38. The method of claim 29 wherein the size of said target nucleic acid is about 100 bp to about 1 kilobase.

39. The method of claim 38 wherein said target nucleic acid and said reference nucleic acid are comparable in length.

40. The method of claim 29 wherein said reference nucleic acid comprises a genomic DNA region.

41. The method of claim 29 wherein said reference nucleic acid is a cDNA transcript, or an oligonucleotide.

42. The method of claim 29 wherein determining the presence or absence of said condition further comprises determining the copy number of said target nucleic acid.

43. The method of claim 29 wherein said nucleic acid binding agent is a DNA binding agent.

44. The method of claim 43 wherein said DNA binding agent is a DNA intercalator.

45. The method of claim 43 wherein said DNA binding agent is selected from the group consisting of EvaGreen, SYBR Green I, PicoGreen, Cyto 9, LC Green, SYBR GreenER, Ethidium bromide, TOTO, YOYO, Bebo, SYTO Green and bexto.

46. The method of claim 45 wherein said DNA binding agent is EvaGreen.

47. The method of claim 29 wherein comparing the melting profiles from said target and references nucleic acid comprises determining the relative amount of signal attributable to said target nucleic acid or to said reference nucleic acid based on their melting profile.

48. The method of claim 47 further comprising comparing the relative amount of the signal attributable to said target nucleic acid and said reference nucleic acid.

49. The method of claim 47 further comprising calculating the ratio between the amount of signal attributable to said target nucleic acid and the amount of signal attributable to said reference nucleic acid.

50. The method of claim 29 wherein said signal is a fluorescent signal, magnetic signal, radioactive signal, Raman signal or an electrochemical signal.

51. A method of determining copy number variation of a target genomic DNA sequence comprising:
providing a sample potentially containing a target genomic DNA sequence
providing a reference nucleic acid and a nucleic acid binding agent, wherein said target genomic DNA sequence said reference nucleic acid exhibit distinct melting profiles;
amplifying the target genomic DNA sequence and the reference nucleic acid using a first set of primers specific for said genomic DNA sequence and a second set of primes specific for said reference nucleic acid sequence;
determining the melting profiles of said amplified target genomic DNA sequence and said amplified reference nucleic acid by monitoring the signal of said binding agent at a plurality of temperatures;
comparing the melting profile of said target nucleic acid to the melting profile of said reference nucleic acid; and
determining the copy number of said target genomic DNA sequence based on said comparison.

52. The method of claim 51 wherein the size of said target nucleic acid is about 100 bp to about 1 kilobase.

53. The method of claim 52 wherein said target nucleic acid and said reference nucleic acid are comparable in length.

54. The method of claim 51 wherein said reference nucleic acid comprises a genomic DNA region.

55. The method of claim 51 wherein said reference nucleic acid is a cDNA transcript, or an oligonucleotide.

56. The method of claim 51 wherein determining the presence or absence of said condition further comprises determining the copy number of said target nucleic acid.

57. The method of claim 51 wherein said nucleic acid binding agent is a DNA binding agent.

58. The method of claim 57 wherein said DNA binding agent is a DNA intercalator.

59. The method of claim 57 wherein said DNA binding agent is selected from the group consisting of EvaGreen, SYBR Green I, PicoGreen, Cyto 9, LC Green, SYBR GreenER, Ethidium bromide, TOTO, YOYO, Bebo, SYTO Green and bexto.

60. The method of claim 59 wherein said DNA binding agent is EvaGreen.

61. The method of claim 51 wherein comparing the melting profiles from said target and references nucleic acid comprises determining the relative amount of signal attributable to said target nucleic acid or to said reference nucleic acid based on their melting profile.

62. The method of claim 61 further comprising comparing the relative amount of the signal attributable to said target nucleic acid and said reference nucleic acid.

63. The method of claim 61 further comprising calculating the ratio between the amount of signal attributable to said target nucleic acid and the amount of signal attributable to said reference nucleic acid.

64. The method of claim 51 wherein said signal is a fluorescent signal, magnetic signal, radioactive signal, Raman signal or an electrochemical signal.

65. A kit comprising:
a nucleic acid binding agent,
a set of primers specific for a reference nucleic acid or alternatively a reference nucleic acid; and
instructions for use of said nucleic acid binding agent, and said set of primers specific or said reference nucleic acid to perform the method describe in claim 1.

66. The kit of claim 65 further comprising a set of primers specific for a target nucleic acid.

67. The kit of claim 66 further compromising a polymerase.

68. The kit of claim 67 further comprising instruction on how to perform amplification of said target nucleic acid and optionally of said reference nucleic acid using said polymerase.

69. The kit claim 65 further comprising a buffer.

70. The method of claim 65 wherein said reference nucleic acid comprises a genomic DNA region.

71. The method of claim 65 wherein said reference nucleic acid is a cDNA transcript, or an oligonucleotide.

72. The method of claim 65 wherein said nucleic acid binding agent is a DNA binding agent.

73. The method of claim 72 wherein said DNA binding agent is a DNA intercalator.

74. The method of claim 65 wherein said DNA binding agent is selected from the group consisting of EvaGreen, SYBR Green I, PicoGreen, Cyto 9, LC Green, SYBR GreenER, Ethidium bromide, TOTO, YOYO, Bebo, SYTO Green and bexto.

75. The method of claim 74 wherein said DNA binding agent is EvaGreen.
